(19) United States
(12) Patent Application Publication
(10) Pub. No.: US 2026/0209245 A1
(43) Pub. Date: Jul. 23, 2026

(54) PROCESS FOR THE PREPARATION OF RELUGOLIX

(71) Applicant: MEDICHEM, S.A., SANT JOAN DESPÍ (ES)

(72) Inventors: JUAN CONTRERAS LASCORZ, CELRÀ (ES); ERNESTO DURÁN LÓPEZ, CASTELLBISBAL (ES)

(21) Appl. No.: 19/139,079

(22) PCT Filed: Dec. 14, 2023
(86) PCT No.: PCT/EP2023/085791

§ 371 Date: Jun. 13, 2025

(30) Foreign Application Priority Data
Dec. 15, 2022 (EP) 22383217.1

(51) Int. Cl. C07D 495/04; B01J 23/44; C07D 333/38

(52) U.S. Cl. CPC C07D 495/04; B01J 23/44; C07D 333/38

(57) ABSTRACT

A process for the preparation of relugolix or a pharmaceutically salt thereof and new intermediates and their use for the preparation of relugolix.

### TECHNICAL FIELD

The present invention relates to a process for the preparation of relugolix or a pharmaceutically salt thereof. It also relates to new intermediates and their use for the preparation of relugolix.

### BACKGROUND ART

Relugolix, represented by the compound of formula (I), is the international commonly accepted name (INN) for 1-(4-(1-(2,6-difluorobenzyl)-5-((dimethylamino)methyl)-3-(6-methoxypyridazin-3-yl)-2,4-dioxo-1,2,3,4-tetrahydrothieno[2,3-d]pyrimidin-6-yl)phenyl)-3-methoxyurea, and has an empirical formula of C₂₉H₂₇F₂N₇O₅S and a molecular weight of 623.6 g/mol.

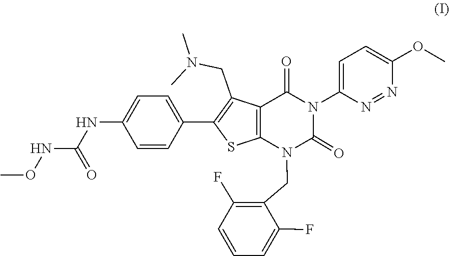

Relugolix is a non-peptide gonadotropin-releasing hormone (GnRH)-receptor antagonist which is used in the treatment of prostate cancer in men and uterine fibroids in women.

International Publication No. WO2004067535A1 describes the preparation of relugolix as shown in Scheme 1. In this case, the compound of formula (II) was coupled with 3-amino-6-chloropyridazine of formula (III) to obtain the compound of formula (IV) which, in the presence of sodium methoxide (NaOMe) in methanol (MeOH), was cyclized in situ, without being isolated, to obtain the compound of formula (V). After a debenzylation step using H₂ in the presence of Pd/C and a subsequent methylation step using methyl iodide (CH₃I), relugolix was obtained. Use of protecting groups such as the benzyl protecting group of the amine moiety is not desirable at industrial scale since it adds unnecessary reaction steps to the overall synthetic process. Moreover, the final methylation step involves the use of highly volatile and mutagenic methyl iodide.

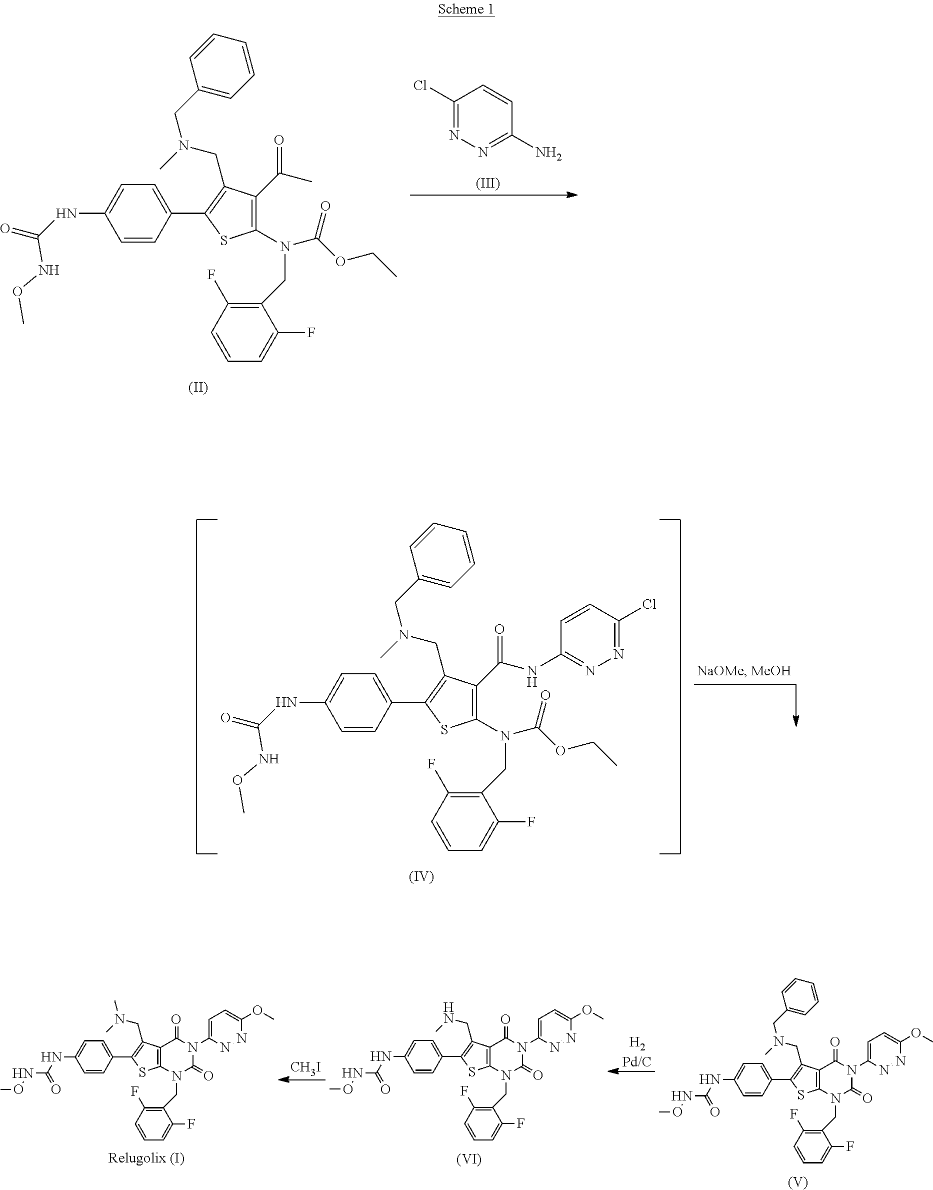

J. Med. Chem. 2011, 54, 4998-5012 describes the preparation of relugolix as shown in Scheme 2. In this case, the compound of formula (VII) was reacted with ethyl chloroformate of formula (VIII-a) to obtain the compound of formula (IX-a). This compound was coupled with 2,6-difluorobenzyl chloride of formula (X) to obtain the compound of formula (XI-a) which after a bromination step using N-bromosuccinimide (NBS) in the presence of 2,2′-azobis(isobutyronitrile) (AIBN) led to the compound (XII-a). The bromo group of compound (XII-a) was replaced by reacting this compound with (2-methoxyethyl)methyl amine of formula (XIII). Then, the nitro compound of formula (XIV) obtained was reduced with H₂ in the presence of Pd/C to obtain the compound of formula (XV). This compound, after a coupling step with methoxyamine in the presence of 1,1′-carbonyldiimidazole (CDI), gave the compound of formula (XVI) which after a hydrolysis reaction in the presence of NaOH gave the compound of formula (XVII). The compound of formula (XVII) was reacted with 3-amino-6-methoxypyridazine of formula (XVIII) and the corresponding amide obtained was cyclized in situ, without being isolated, in the presence of sodium methoxide (NaOMe) in methanol (MeOH) to obtain the compound of formula (XIX). By reacting this compound with 1-chloroethylchloroformate and subsequently adding dimethylamine, relugolix was obtained.

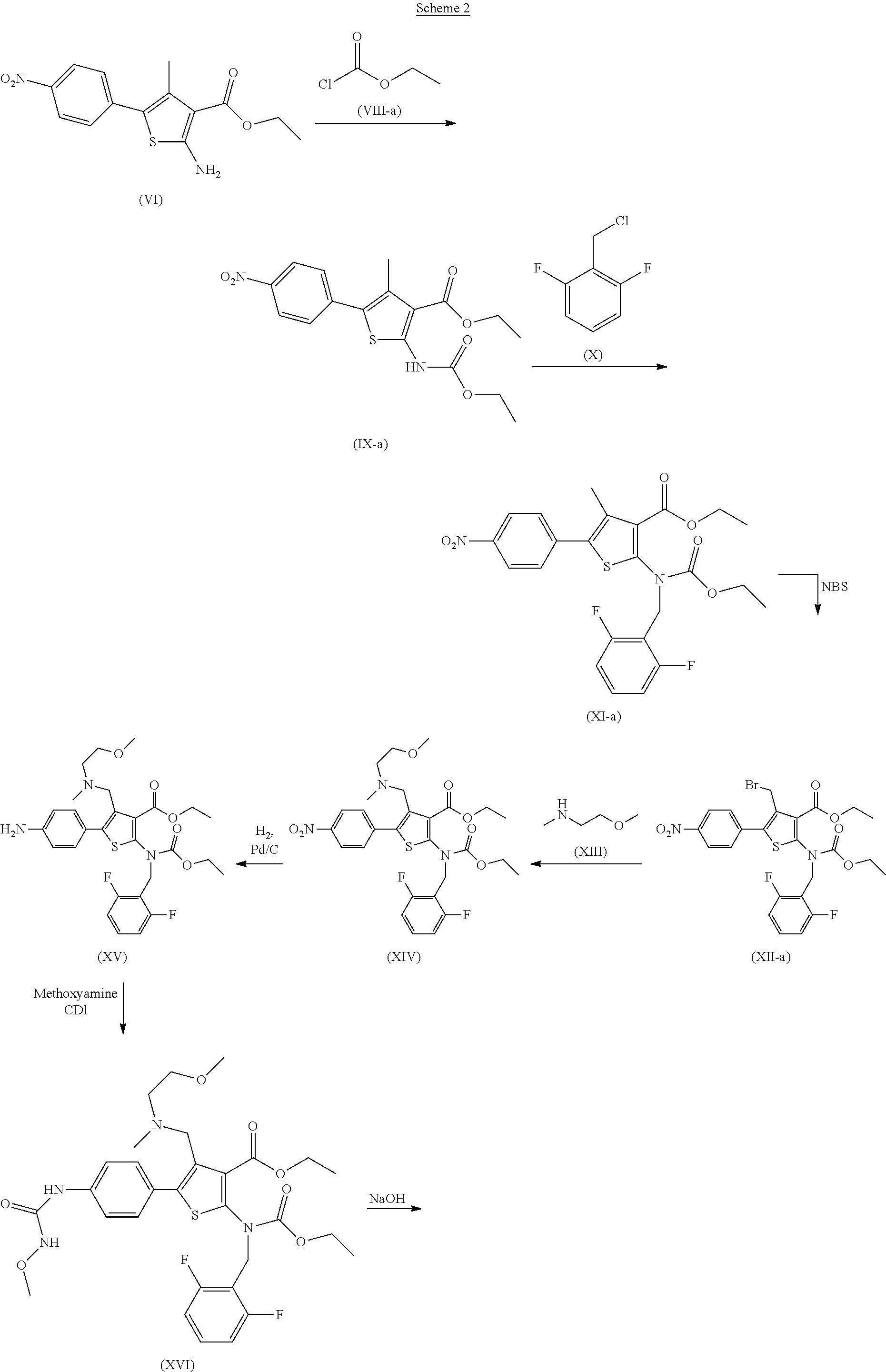

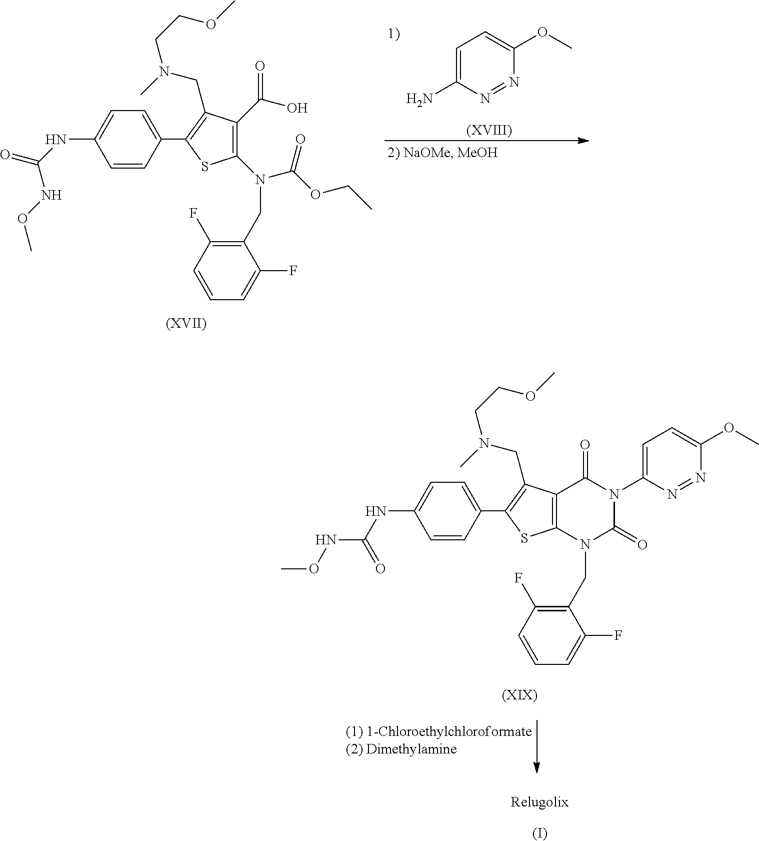

International Publication No. WO2014051164A1 discloses an alternative synthetic approach to obtain relugolix. In this case, as shown in Scheme 3, the bromo group of the compound of formula (XII-a), obtained as disclosed above in Scheme 2, was replaced by reacting with dimethylamine. The compound of formula (XX-a) thus obtained was hydrolyzed in the presence of KOH to obtain the compound of formula (XXI). This compound was reacted with 3-amino-6-methoxypyridazine of formula (XVIII) to obtain the corresponding amide of formula (XXII-a), which was cyclized in the presence of sodium methoxide (NaOMe) in methanol (MeOH) to obtain the compound of formula (XXIII) which was reduced with H₂ in the presence of Pd/C to obtain the compound of formula (XXIV). After a subsequent coupling step with methoxyamine in the presence of 1,1′-carbonyldiimidazole (CDI) relugolix was obtained.

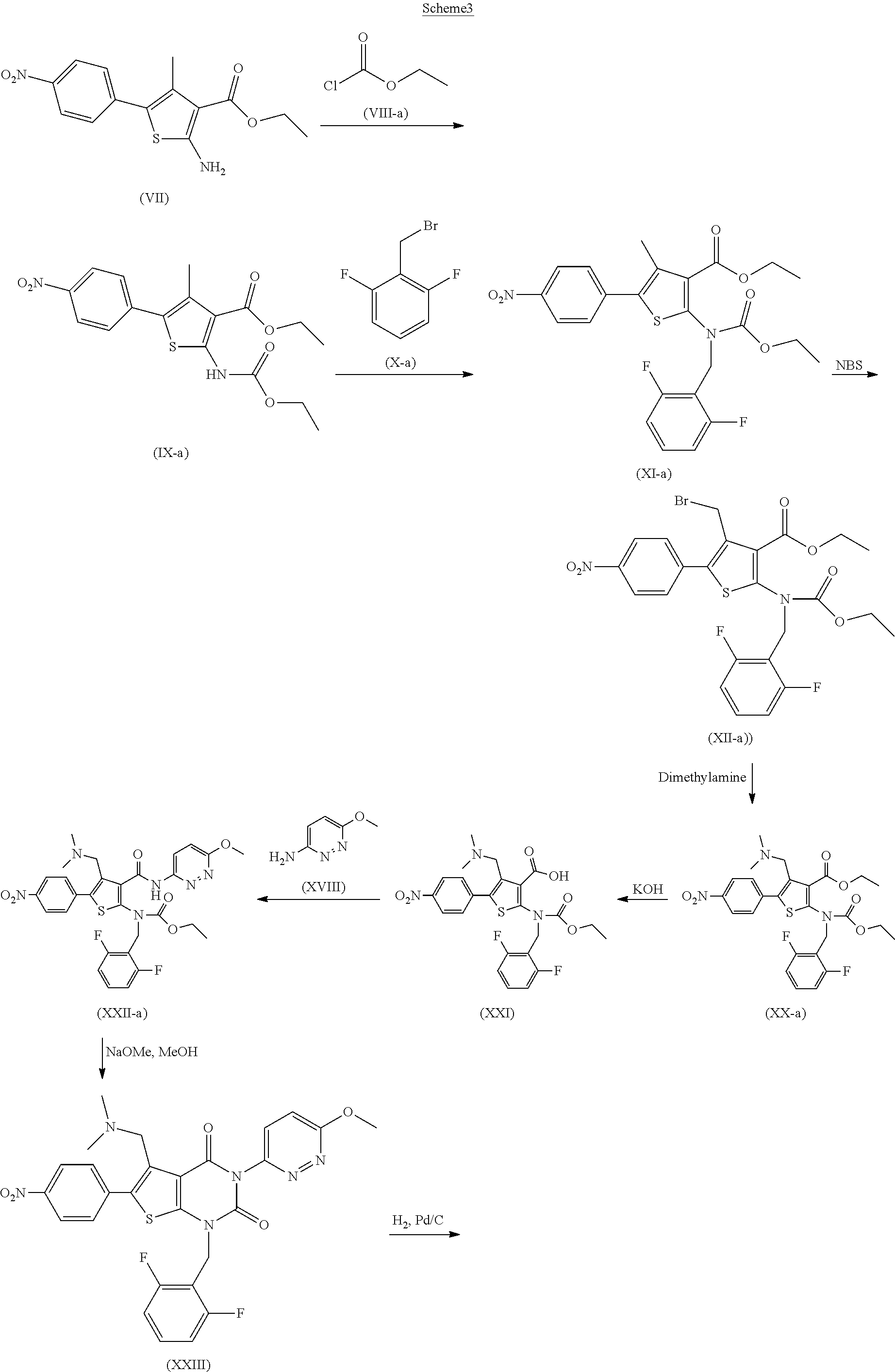

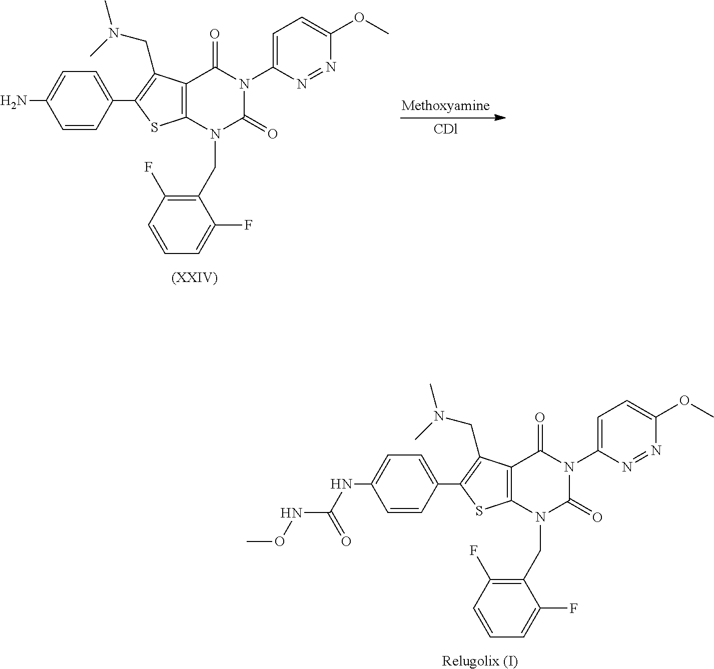

Chinese patent applications CN112745304A and CN112321602A disclose an alternative synthetic approach to obtain relugolix as shown in Scheme 4. In this case the compound of formula (XXII) was transformed using a reducing agent into the compound of formula (XXV). This compound was reacted with methoxyamine in the presence of a coupling agent to give the compound of formula (XXVI) which after a cyclization reaction gave relugolix. In Scheme 4, R is a substituted or unsubstituted C₁-C₆ saturated or unsaturated alkyl or a C₁-C₆ alkylaryl, preferably methyl, ethyl, propyl, allyl, chloroethyl, benzyl as described in CN112745304A, and a C₃₋₇-alkyl group or an aryl group as described in CN112321602A.

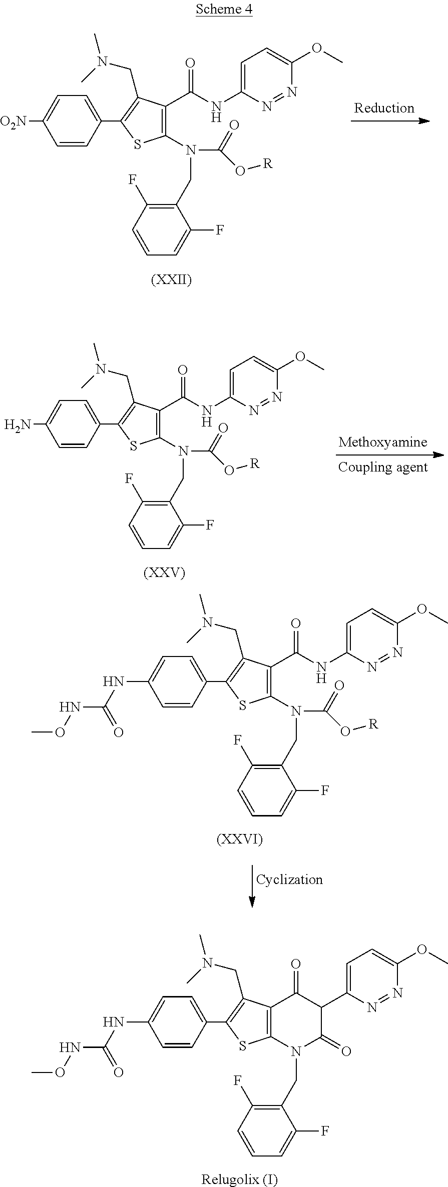

Chinese patent application CN111333633A discloses an alternative approach to obtain relugolix. In this case, as shown in Scheme 5, the nitro compound of formula (XX), obtained by replacing the halogen group of the compound (XII) by dimethylamine, was reduced with H₂ in the presence of Pd/C to obtain the amine compound of formula (XXVII) which after reaction with methoxyamine in the presence of a coupling agent gave the compound of formula (XXVIII). This compound, after a hydrolysis reaction in the presence of a base, was converted into the compound of formula (XXIX) which was reacted with 3-amino-6-methoxypyridazine of formula (XVIII) to obtain the corresponding amide of formula (XXVI), which after a cyclization reaction gave relugolix. According to CN111333633A, the R and R₁ groups in Scheme 5 are independently a C₁-C₆ straight chain or branched alkyl, preferably R is ethyl or isobutyl and R₁ is ethyl, and the X group represents Cl, Br or I.

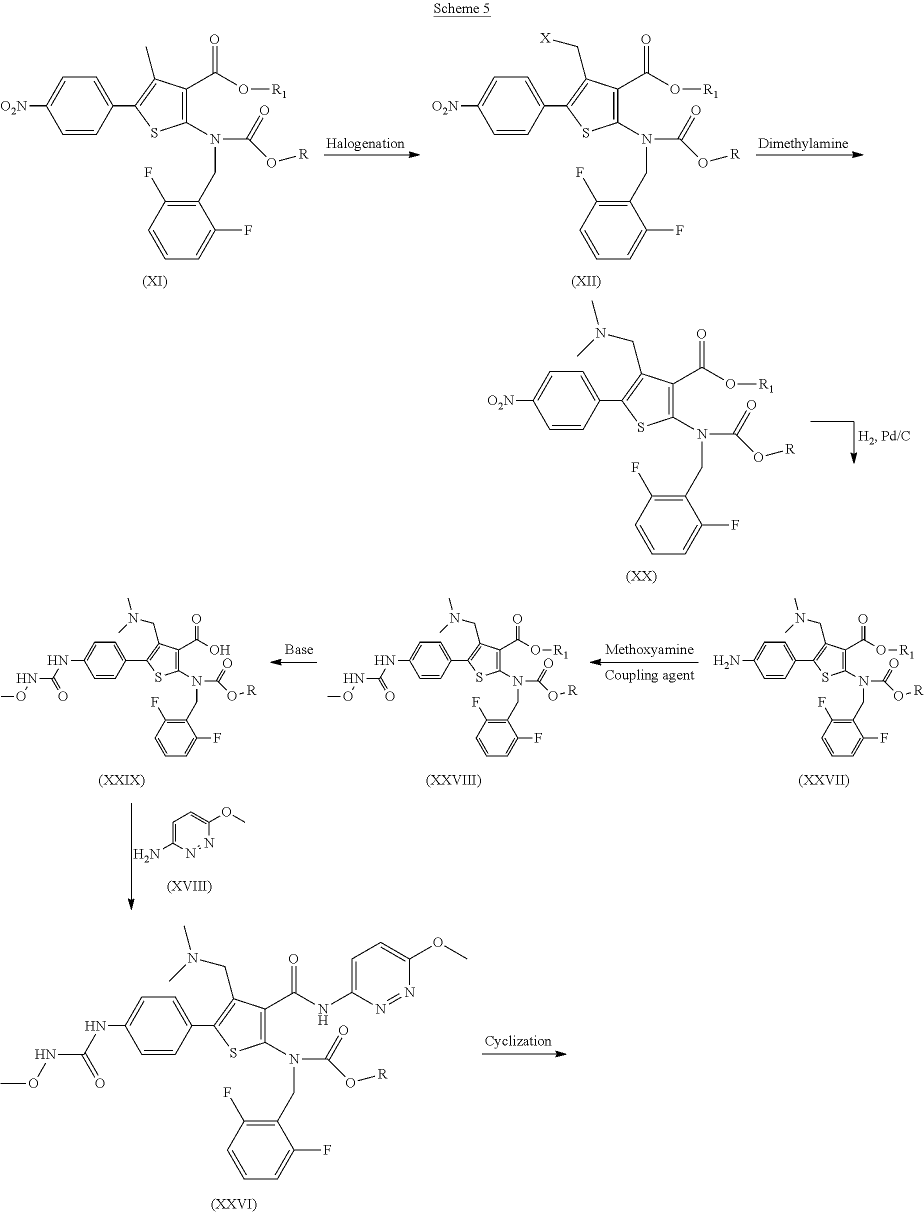

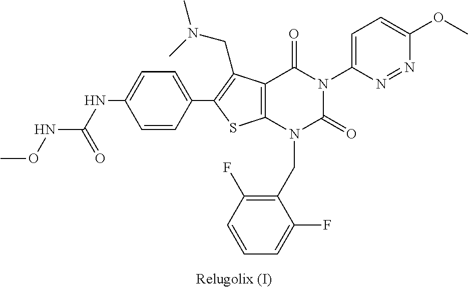

Indian patent application IN202021052222A discloses the preparation of relugolix from compound of formula (XX) according to the same synthetic strategy as shown in Scheme 5 wherein R and R¹ independently are C₁₋₅ alkyl, selected from methyl, ethyl, n-propyl, isopropyl, n-butyl, and t-butyl.

Chinese patent application CN113501830A discloses the preparation of relugolix from compound of formula (XX) according to the same synthetic strategy as shown in Scheme 5 wherein R and R₁ independently are ethyl.

Chinese patent application CN113444105A discloses the preparation of relugolix from compound of formula (XXVII) according to the same synthetic strategy as shown in Scheme 5 wherein R and R₁ independently are ethyl.

Chinese patent application CN114685468A discloses the preparation of relugolix from compound of formula (XX) according to the same synthetic strategy as shown in Scheme 5 wherein R is benzyl or substituted benzyl, C₇-10 straight or branched chain alkyl, preferably benzyl or octyl, and R₁ is ethyl.

Other synthetic approaches to prepare relugolix are disclosed in Chinese patent applications CN114621249A, CN114790189A, CN114230576A, CN114031626A, CN113135934A, CN113429423A, CN111423452A, CN110194776A, CN115073490A and in the international Publications No. WO2019020102A1 and WO2022214645A1.

Some of the above prior art documents disclose the preparation of relugolix in low yield, which implies the increase of the cost of the relugolix process and the pharmaceutical compositions containing this active ingredient, which already resulted in expensive medicines. Moreover, some of the above prior art documents disclose the use of explosive and difficult to handle reagents and require purifications by column chromatography.

Therefore, from what is known in the prior art, there is a need in developing a feasible and scalable process for the synthesis of relugolix, or a pharmaceutically acceptable salt thereof, in high purity and industrially acceptable yields. The present invention fulfils this and related needs.

### SUMMARY OF THE INVENTION

The inventors have found a new process for the preparation of relugolix or a pharmaceutically acceptable salt thereof that overcomes and/or minimizes some of the drawbacks of the processes disclosed in the prior art. The new process allows obtaining these compounds with unexpectedly high overall yields, and at the same time with a high purity. The process is easy to scale-up to an industrial scale, allows the manufacture of relugolix with a high overall yield and with the required purity for pharmaceutical uses without the need of extensive purification steps, and consequently is more cost-effective and produces less residues than the already known processes.

In a first aspect of the invention there is provided a process for the preparation of relugolix of formula (I), or a pharmaceutically acceptable salt thereof, which comprises:
a) cyclizing a compound of formula (XXVI-b) in the presence of a base selected from the group consisting of 1,5,7-triazabicyclo[4.4.0]dec-5-ene (TBD), 7-methyl-1,5,7-triazabicyclo[4.4.0]dec-5-ene (MTBD), 1,8-diazabicyclo[5.4.0]undec-7-ene (DBU), 1,5-diazabicyclo[4.3.0]non-5-ene (DBN), and 1,1,3,3-tetramethylguanidine (TMG) in the presence of a suitable solvent to obtain relugolix, and

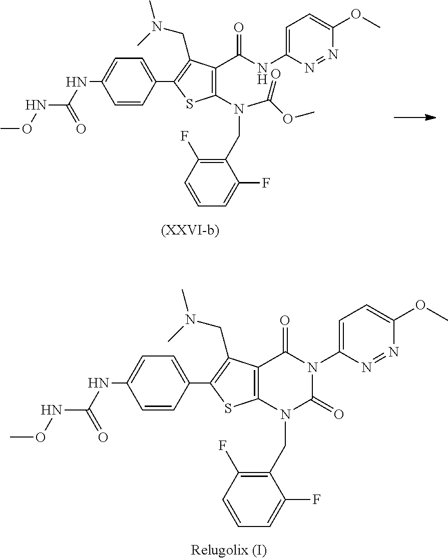

b) optionally converting the relugolix of formula (I) into a pharmaceutically acceptable salt thereof.

A second aspect of the invention relates to a compound of formula (XXVII-b·HCl)

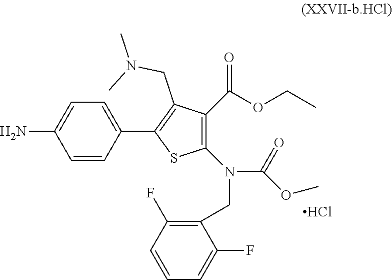 [SMILES: CCOC(=O)c1c(N(Cc2c(F)cccc2F)C(=O)OC)sc(-c2ccc(N)cc2)c1CN(C)C ; Cl]

Finally, another aspect of the invention relates to a compound of formula (XX-b·oxalate).

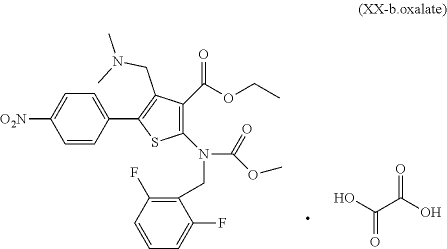 [SMILES: CCOC(=O)c1c(N(Cc2c(F)cccc2F)C(=O)OC)sc(-c2ccc([N+](=O)[O-])cc2)c1CN(C)C ; "·" ; O=C(O)C(=O)O]

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly throughout the specification and claims.

For the purposes of the present invention, any ranges given include both the lower and the upper end-points of the range. Ranges and values given, such as temperatures, times, and the like, should be considered approximate, unless specifically stated.

The term “about” as used herein refers to a range of values ±10% of a specified value. For example, the expression “about 10” includes ±10% of 10, i.e., from 9 to 11.

The term “room temperature” refers to a temperature of the environment, without heating or cooling, and is generally from 20° C. to 25° C.

It is noted that, as used in this specification and the appended claims, the singular forms “a”, “an”, and “the” include plural referents unless the context clearly dictates otherwise.

As mentioned above, the first aspect of the present invention provides a process for the preparation of relugolix of formula (I), or a pharmaceutically acceptable salt therefor, which comprises:
a) cyclizing a compound of formula (XXVI-b) in the presence of a base selected from the group consisting of 1,5,7-triazabicyclo[4.4.0]dec-5-ene (TBD), 7-methyl-1,5,7-triazabicyclo[4.4.0]dec-5-ene (MTBD), 1,8-diazabicyclo[5.4.0]undec-7-ene (DBU), 1,5-diazabicyclo[4.3.0]non-5-ene (DBN), and 1,1,3,3-tetramethylguanidine (TMG) in the presence of a suitable solvent to obtain relugolix, and

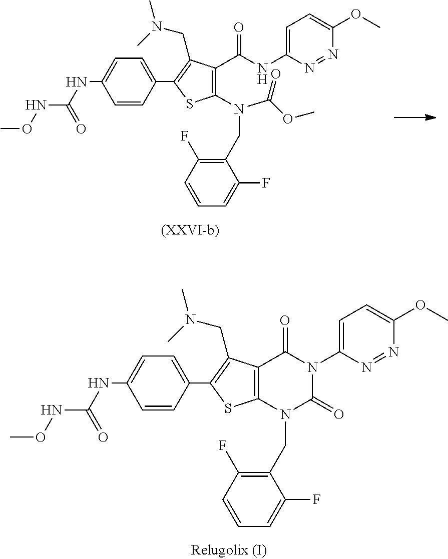

b) optionally converting the relugolix of formula (I) into a pharmaceutically acceptable salt thereof.

Step a) involves the cyclization of a compound of formula (XXVI-b) in the presence of a base selected from the group consisting of 1,5,7-triazabicyclo[4.4.0]dec-5-ene (TBD), 7-methyl-1,5,7-triazabicyclo[4.4.0]dec-5-ene (MTBD), 1,8-diazabicyclo[5.4.0]undec-7-ene (DBU), 1,5-diazabicyclo[4.3.0]non-5-ene (DBN), and 1,1,3,3-tetramethylguanidine (TMG).

In an embodiment of the present invention, the base used in step (a) is 1,8-diazabicyclo[5.4.0]undec-7-ene (DBU).

The amount of base used in step (a), preferably 1,8-diazabicyclo[5.4.0]undec-7-ene (DBU), is from 0.05 equivalents to 6 equivalents, preferably from 0.1 equivalents to 5 equivalents, more preferably from 0.1 equivalents to 2.5 equivalents, based on the compound of formula (XXVI-b).

Non-limiting examples of suitable solvents that can be used in step a), which can be used alone or as a mixture of solvents, include water; alcohols such as methanol, ethanol, isopropanol, n-propanol, n-butanol, isobutanol, sec-butanol, or tert-butanol; ketones such as acetone, methyl ethyl ketone, or methyl isobutyl ketone; ethers such as tetrahydrofuran, dioxane, diisopropylether, diethylether, 2-methyltetrahydrofuran, cyclopentyl methyl ether, or methyl tert-butyl ether; esters such as ethyl acetate, methyl acetate, isopropyl acetate, n-propyl acetate, n-butyl acetate, isobutyl acetate, sec-butyl acetate, or tert-butyl acetate; halogenated solvents such as dichloromethane, chloroform, tetrachloromethane, dichloroethane, chlorobenzene or dichlorobenzene; polar aprotic solvents such as N,N-dimethylformamide, acetonitrile, N,N-dimethylacetamide, N-methyl-2-pyrrolidone or dimethylsulfoxide; hydrocarbon aliphatic solvents such as methylcyclohexane, cyclohexane, heptane, or hexane; aromatic hydrocarbon solvents such as toluene, benzene, o-xylene, m-xylene, or p-xylene.

Preferably, the reaction takes place in the presence of dimethylsulfoxide.

Preferably, the reaction is carried out at a temperature in the range from −10° C. to 70° C., preferably from 0° C. to 60° C., and more preferably from 20° C. to 50° C.

The relugolix of formula (I) obtained in step a) can be isolated from the reaction mixture by any method known in the art. Preferably, the relugolix is isolated by filtration.

The relugolix of formula (I) obtained in step a) can be purified by any method known in the art such as crystallization or slurring. Preferably, relugolix may be purified by crystallization.

Optionally, the relugolix of formula (I) obtained in step a) may be converted into a pharmaceutically salt thereof. The salt can be purified by any method known in the art such as crystallization or slurring.

The term “pharmaceutically acceptable salt” refers to any salt that possesses the desired pharmacological activity of the parent compound and that is formed from non-toxic pharmaceutically acceptable acids, that include, but are not limited to, organic acids and/or inorganic acids. Such acids include for example hydrochloric, hydrobromic, sulfuric, perchloric, phosphoric, nitric, methanesulfonic, p-toluenesulfonic, benzenesulfonic, acetic, fumaric, trifluoroacetic, propionic, glycolic, lactic, oxalic, malonic, malic, maleic, tartaric, succinic, citric, benzoic, cinnamic, mandelic, and salicylic acid.

In a particular embodiment, optionally in combination with one or more features of the various embodiments described above or below, the process, previously to step a), further comprises the steps of:
i) coupling a compound of formula (XXVII-b·HCl) with methoxyamine or a salt thereof in the presence of a coupling agent and a suitable solvent to give a compound of formula (XXVIII-b),

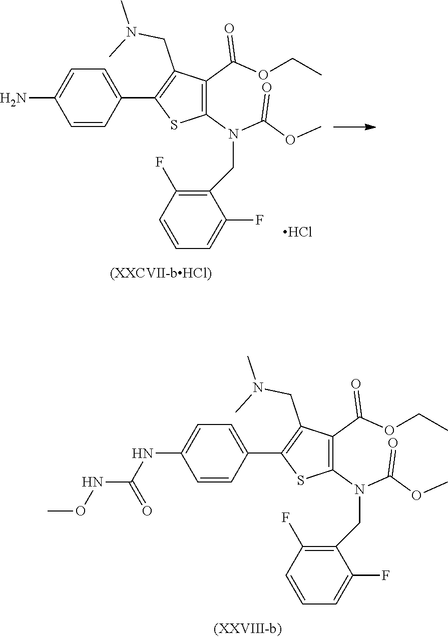

ii) hydrolyzing the compound of formula (XXVIII-b) obtained in step (i) to give a compound of formula (XX-b), and

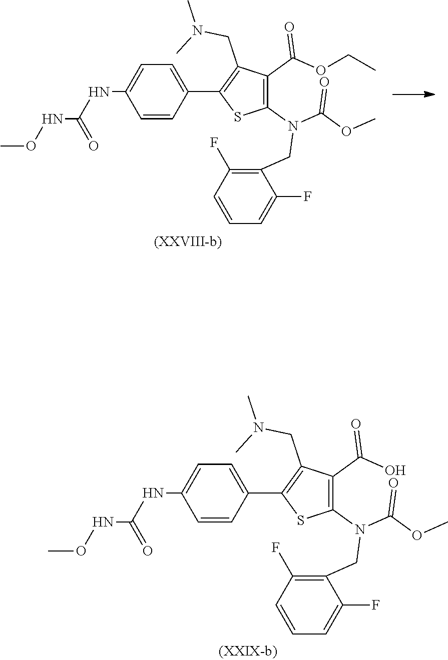

ii) coupling the compound of formula (XXXIX-b) obtained in step (ii) with 3-amino-6-methoxypyridazine of formula (XVIII) in the presence of a coupling agent and in a suitable solvent to give a compound of formula (XXVI-b).

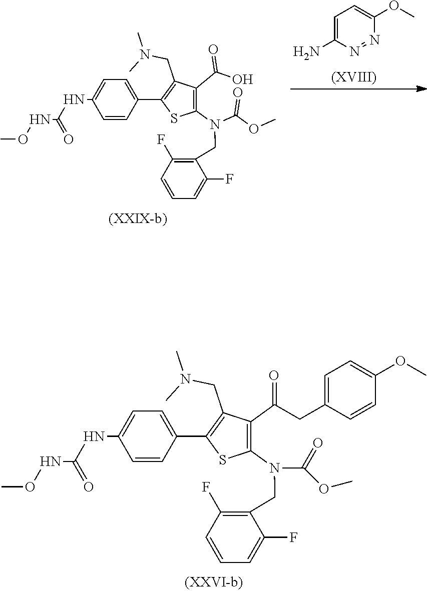

The coupling step i) is carried out in the presence of methoxyamine or a salt thereof. Preferably, the coupling step i) is carried out using the hydrochloride salt of methoxyamine (methoxyamine·HCl).

The amount of the methoxyamine, preferably the hydrochloride salt of methoxyamine, is from 0.5 equivalents to 8 equivalents, and preferably from 1 equivalent to 5 equivalents, based on the compound of formula (XXVII-b·HCl).

Non-limiting examples of suitable coupling agents that can be used in the coupling step i) are propylphosphonic anhydride (T3P), 1,1′-carbonyldiimidazole (CDI), 1,1′-carbonylditetrazole (CDT), N,N′-disuccinimidyl carbonate (DSC), bis(trichloromethyl) carbonate (BTC), N,N′-dicyclohexylcarbodiimide, 1-ethyl-3-(3-dimethylaminopropyl)carbodiimide, 2-chloro-4,6-dimethoxy-1,3,5-triazine (CDMT), 1-[bis(dimethylamino)methylene]-1H-1,2,3-triazolo[4,5-b]pyridinium 3-oxide hexafluorophosphate (HATU), triphosgene, acid chlorides such as oxalyl chloride, pivaloyl chloride, and the like, alkyl chloroformates such as methyl chloroformate and the like, and bis(1-benzotriazolyl)methanethione.

Preferably, the coupling agent used in step i) is 1,1′-carbonyldiimidazole (CDI).

The amount of the coupling agent, preferably 1,1′-carbonyldiimidazole (CDI), is from 0.5 equivalents to 8 equivalents, and preferably from 1 equivalent to 5 equivalents, based on the compound of formula (XXVII-b·HCl).

The coupling step i) is carried out in the presence of a base which is not particularly limited. Preferably, the base is selected from the group consisting of triethylamine, trimethylamine, diethylamine, diethanolamine, diisopropylethylamine, pyridine, dimethylaminopyridine (DMAP), dicyclohexylamine, triethanolamine, meglumine, ethylenediamine, picoline, quinoline, 1,8-diazabicyclo[5.4.0]undec-7-ene (DBU), sodium carbonate, potassium carbonate, sodium bicarbonate, potassium bicarbonate, and mixtures thereof.

Preferably, the base used in step i) is a mixture of triethylamine and sodium carbonate.

Non-limiting examples of suitable solvents that can be used in step i), which can be used alone or as a mixture of solvents, include ketones such as acetone, methyl ethyl ketone, or methyl isobutyl ketone; ethers such as tetrahydrofuran, dioxane, diisopropylether, diethylether, 2-methyltetrahydrofuran, cyclopentyl methyl ether, or methyl tert-butyl ether; esters such as ethyl acetate, methyl acetate, isopropyl acetate, n-propyl acetate, n-butyl acetate, isobutyl acetate, sec-butyl acetate, or tert-butyl acetate; halogenated solvents such as dichloromethane, chloroform, tetrachloromethane, dichloroethane, chlorobenzene, or dichlorobenzene; polar aprotic solvents such as N,N-dimethylformamide, acetonitrile, N,N-dimethylacetamide, N-methyl-2-pyrrolidone, or dimethylsulfoxide; hydrocarbon aliphatic solvents such as methylcyclohexane, cyclohexane, heptane, or hexane; aromatic hydrocarbon solvents such as toluene, benzene, o-xylene, m-xylene, or p-xylene.

Preferably, the coupling step i) is carried out using acetonitrile as solvent.

The coupling step i) can be carried out at a temperature in the range from 20° C. to 80° C., preferably from 40° C. to 60° C.

The compound of formula (XXVIII-b) can be isolated prior to the hydrolysis step ii), or alternatively, it can be used in the next step ii) without isolation.

The compound of formula (XXVIII-b) may be purified by any method known in the art before used in the next processing step.

The hydrolysis step ii) can be carried out in the presence of a base or an acid which are not particularly limited. Non-limiting examples of suitable bases are sodium hydroxide, potassium hydroxide, lithium hydroxide, sodium methoxide and sodium ethoxide. Non-limiting examples of suitable acids are hydrochloric acid, sulfuric acid, and phosphoric acid.

Preferably, the hydrolysis agent used in step ii) is sodium hydroxide.

The hydrolysis step ii) can be carried out at a temperature in the range from 20° C. to 60° C., preferably from 30° C. to 50° C.

The compound of formula (XXIX-b) can be isolated prior to the coupling step iii), or alternatively, it can be used in the step iii) without isolation.

The compound of formula (XXIX-b) may be purified by any method known in the art before used in the next processing step.

Non-limiting examples of suitable solvents that can be used in step ii), which can be used alone or as a mixture of solvents, include water; alcohols such as methanol, ethanol, isopropanol, n-propanol, n-butanol, isobutanol, sec-butanol or tert-butanol; ketones such as acetone, methyl ethyl ketone, or methyl isobutyl ketone; ethers such as tetrahydrofuran, dioxane, diisopropylether, diethylether, 2-methyltetrahydrofuran, cyclopentyl methyl ether, or methyl tert-butyl ether; halogenated solvents such as dichloromethane, chloroform, tetrachloromethane, dichloroethane, chlorobenzene or dichlorobenzene; polar aprotic solvents such as N,N-dimethylformamide, acetonitrile, N,N-dimethylacetamide, N-methyl-2-pyrrolidone, or dimethylsulfoxide; hydrocarbon aliphatic solvents such as methylcyclohexane, cyclohexane, heptane or hexane; aromatic hydrocarbon solvents such as toluene, benzene, o-xylene, m-xylene, or p-xylene.

Preferably, the hydrolysis step ii) is carried out in the presence of acetone and water.

The coupling step iii) of a compound of formula (XXIX-b) with a compound of formula (XVIII) is carried out in the presence of a coupling agent in a suitable solvent.

Non-limiting examples of suitable coupling agents that can be used in the coupling step iii) are propylphosphonic anhydride (T3P), 1,1′-carbonyldiimidazole (CDI), 1,1′-carbonylditetrazole (CDT), N,N′-disuccinimidyl carbonate (DSC), bis(trichloromethyl) carbonate (BTC), N,N′-dicyclohexylcarbodiimide, 1-ethyl-3-(3-dimethylaminopropyl)carbodiimide, 2-chloro-4,6-dimethoxy-1,3,5-triazine (CDMT), (1-[bis(dimethylamino)methylene]-1H-1,2,3-triazolo[4,5-b]pyridinium 3-oxide hexafluorophosphate (HATU), triphosgene, acid chlorides such as oxalyl chloride, pivaloyl chloride, and the like, alkyl chloroformates such as methyl chloroformate, and the like, and bis(1-benzotriazolyl)methanethione.

Preferably, the coupling agent used in step iii) is propylphosphonic anhydride (T3P).

The amount of the coupling agent, preferably propylphosphonic anhydride (T3P), is from 0.5 equivalents to 8 equivalents, and preferably from 1 equivalent to 5 equivalents, based on the compound of formula (XXIX-b).

Non-limiting examples of suitable solvents that can be used in the coupling step iii), which can be used alone or as a mixture of solvents, include ketones such as acetone, methyl ethyl ketone, or methyl isobutyl ketone; ethers such as tetrahydrofuran, dioxane, diisopropylether, diethylether, 2-methyltetrahydrofuran, cyclopentyl methyl ether, or methyl tert-butyl ether; esters such as ethyl acetate, methyl acetate, isopropyl acetate, n-propyl acetate, n-butyl acetate, isobutyl acetate, sec-butyl acetate, or tert-butyl acetate; halogenated solvents such as dichloromethane, chloroform, tetrachloromethane, dichloroethane, chlorobenzene, or dichlorobenzene; polar aprotic solvents such as N,N-dimethylformamide, acetonitrile, N,N-dimethylacetamide, N-methyl-2-pyrrolidone, or dimethylsulfoxide; hydrocarbon aliphatic solvents such as methylcyclohexane, cyclohexane, heptane, or hexane; aromatic hydrocarbon solvents such as toluene, benzene, o-xylene, m-xylene, or p-xylene.

Preferably, the solvent used in the coupling step iii) is ethyl acetate.

The coupling step iii) is carried out in the presence of a base which is not particularly limited. Preferably, the base is selected from the group consisting of triethylamine, trimethylamine, diethylamine, diethanolamine, diisopropylethylamine, pyridine, dimethylaminopyridine (DMAP), dicyclohexylamine, triethanolamine, meglumine, ethylenediamine, picoline, quinoline, 1,8-diazabicyclo[5.4.0]undec-7-ene (DBU), sodium carbonate, potassium carbonate, sodium bicarbonate, potassium bicarbonate, and mixtures thereof.

Preferably, the base used in the coupling step iii) is diisopropylethylamine.

The coupling step iii) can be carried out at a temperature in the range from 20° C. to 80° C., preferably from 30° C. to 50° C.

The compound of formula (XXVI-b) can be isolated prior to step a), or alternatively, it can be used in step a) without isolation.

The compound of formula (XXVI-b) may be purified by any method known in the art before used in the next processing step.

In another particular embodiment, optionally in combination with one or more features of the various embodiments described above or below, the process, previously to step i), further comprises the steps of:
iv) neutralizing a compound of formula (XX-b·oxalate) to obtain a compound of formula (XX-b), and

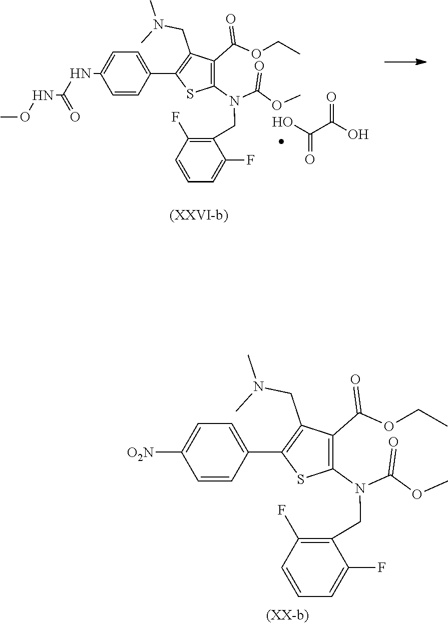

v) reducing the compound of formula (XX-b) obtained in step (iv) to obtain a compound of formula (XXVII-b), which is further converted to a compound of formula (XXVII-b·HCl)

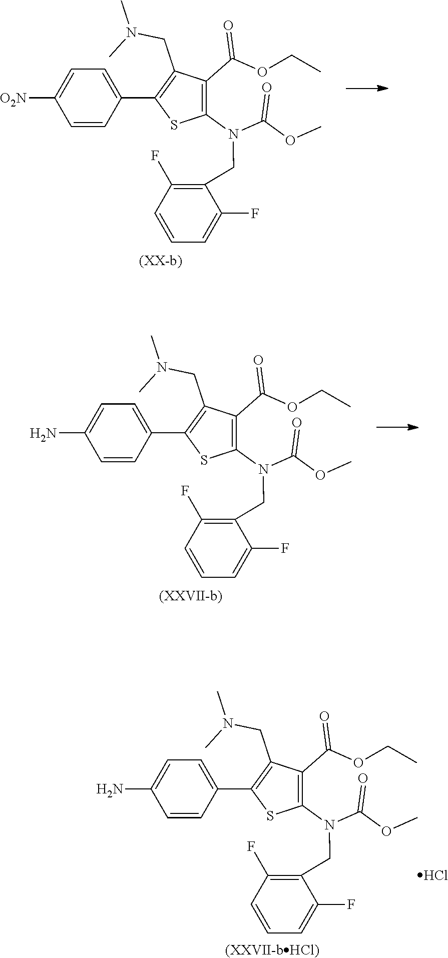 [SMILES: CCOC(=O)c1c(N(Cc2c(F)cccc2F)C(=O)OC)sc(-c2ccc([N+](=O)[O-])cc2)c1CN(C)C ; CCOC(=O)c1c(N(Cc2c(F)cccc2F)C(=O)OC)sc(-c2ccc(N)cc2)c1CN(C)C ; CCOC(=O)c1c(N(Cc2c(F)cccc2F)C(=O)OC)sc(-c2ccc(N)cc2)c1CN(C)C ; Cl]

The neutralization step (iv) may be carried out by treating a compound of formula (XX-b·oxalate) with a suitable base such as sodium hydroxide, sodium carbonate, sodium bicarbonate, potassium hydroxide, potassium carbonate, potassium bicarbonate, lithium hydroxide, lithium carbonate and lithium bicarbonate.

Preferably, the neutralization agent used in step iv) is potassium carbonate.

The compound of formula (XX-b) can be isolated prior to step v), or alternatively, it can be used in step v) without isolation.

The compound of formula (XX-b) may be purified by any method known in the art before used in the next processing step.

Preferably, the reduction step (v) comprises the catalytic hydrogenation of a compound of formula (XX-b) with hydrogen in the presence of a catalyst.

The term “catalytic hydrogenation” refers to the treatment with hydrogen in the presence of a catalyst. The catalyst can be homogeneous or heterogeneous. The catalyst typically comprises a transition metal catalyst. The transition metal catalyst is preferably selected from the group comprising palladium, platinum, nickel and rhodium catalysts or mixtures thereof. Preferably, the catalyst is palladium on charcoal (Pd/C).

The catalytic hydrogenation of a compound of formula (XX-b) is carried out at a temperature in the range from 0° C. to 50° C., preferably from 15° C. to 45° C., and more preferably from 20° C. to 40° C.

Preferably, the amount of catalyst used is from 0.5% w/w to less or equal than 10% w/w, preferably from 1% to 5%, based on the amount of the compound of formula (XX-b) that has been used in the preparation of the compound of formula (XXVII-b).

Preferably, the catalytic hydrogenation is carried out under a hydrogen pressure from about atmospheric pressure to 6 bar, preferably in the range from 2 to 4 bar.

Preferably, the catalytic hydrogenation takes place at a temperature in the range from 10° C. to 60° C., more preferably from 20° C. to 50° C., even more preferably at about 30° C.

Non-limiting examples of suitable solvents that can be used in the reduction step (v), which can be used alone or as a mixture of solvents, include water; alcohols such as methanol, ethanol, isopropanol, n-propanol, n-butanol, isobutanol, sec-butanol, or tert-butanol; ethers such as tetrahydrofuran, dioxane, diisopropylether, diethylether, 2-methyltetrahydrofuran, cyclopentyl methyl ether, or methyl tert-butyl ether; esters such as ethyl acetate, methyl acetate, isopropyl acetate, n-propyl acetate, n-butyl acetate, isobutyl acetate, sec-butyl acetate, or tert-butyl acetate; polar aprotic solvents such as N,N-dimethylformamide, acetonitrile, N,N-dimethylacetamide, N-methyl-2-pyrrolidone, or dimethylsulfoxide; hydrocarbon aliphatic solvents such as methylcyclohexane, cyclohexane, heptane, or hexane; aromatic hydrocarbon solvents such as toluene, benzene, o-xylene, m-xylene, or p-xylene.

The compound of formula (XXVII-b) which is formed after the hydrogenation step can be isolated prior to the conversion to the hydrochloride salt or alternatively it can be converted in situ, without being isolated.

The compound of formula (XXVII-b) may be purified by any method known in the art before used in the next processing step.

The conversion of the compound of formula (XXVII-b) into the compound of formula (XXVII-b·HCl) is carried out by reacting the obtained compound of formula (XXVII-b) with hydrogen chloride (HCl) by any method known in the art.

Non-limiting examples of suitable solvents that can be used in the conversion of the compound of formula (XXVII-b) into the compound of formula (XXVII-b·HCl), which can be used alone or as a mixture of solvents, include water; alcohols such as methanol, ethanol, isopropanol, n-propanol, n-butanol, isobutanol, sec-butanol, or tert-butanol; aromatic hydrocarbon solvents such as benzene or nitrobenzene; ketones such as acetone, methyl ethyl ketone, or methyl isobutyl ketone; ethers such as tetrahydrofuran, dioxane, diisopropylether, diethylether, 2-methyltetrahydrofuran, cyclopentyl methyl ether, or methyl tert-butyl ether; esters such as ethyl acetate, methyl acetate, isopropyl acetate, n-propyl acetate, n-butyl acetate, isobutyl acetate, sec-butyl acetate, or tert-butyl acetate; halogenated solvents such as dichloromethane, chloroform, tetrachloromethane, dichloroethane, chlorobenzene, or dichlorobenzene; polar aprotic solvents such as N,N-dimethylformamide, acetonitrile, N,N-dimethylacetamide, N-methyl-2-pyrrolidone, or dimethylsulfoxide; hydrocarbon aliphatic solvents such as methylcyclohexane, cyclohexane, heptane, or hexane. Preferably, the solvent is ethyl acetate and isopropanol.

The compound of formula (XXVII-b·HCl) thus obtained, may be purified by any method known in the art before used in the next processing step.

In another particular embodiment, optionally in combination with one or more features of the various embodiments described above or below, the process, previously to step iv), further comprises the steps of:
vi) reacting a compound of formula (VII) with methyl chloroformate of formula (VIII-b) to give a compound of formula (IX-b),
vii) reacting a compound of formula (IX-b) obtained in step (vi) with 2,6-difluorobenzyl bromide of formula (X-a) to give a compound of formula (XI-b),
viii) brominating the compound of formula (XI-b) obtained in step (vii) with N-bromosuccinimide (NBS) to obtain a compound of formula (XII-b),
ix) reacting the compound of formula (XII-b) obtained in step (viii) with dimethylamine to give a compound of formula (XX-b), and
x) reacting the compound of formula (XX-b) obtained in step ix) with oxalic acid to obtain the compound of formula (XX-b·oxalate).

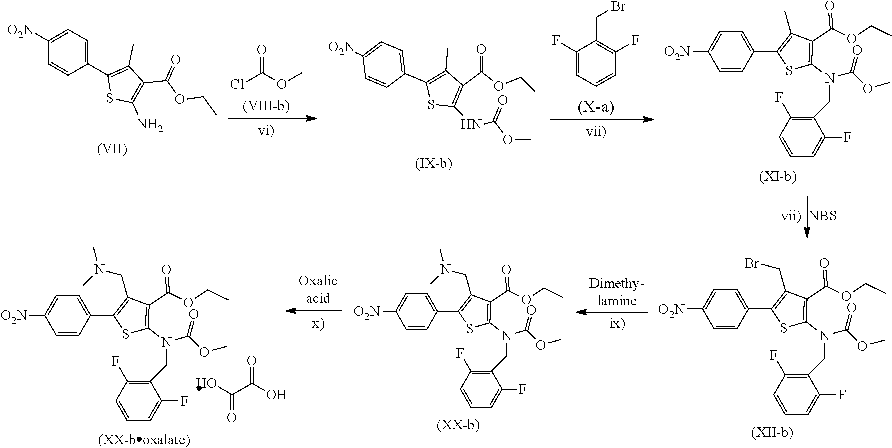

The step vi) is carried out in the presence of methyl chloroformate (VIII-b) with a suitable solvent.

Non-limiting examples of suitable solvents that can be used in step vi), which can be used alone or as a mixture of solvents, include aromatic hydrocarbon solvents such as toluene, benzene, o-xylene, m-xylene, p-xylene, or nitrobenzene; ketones such as acetone, methyl ethyl ketone, or methyl isobutyl ketone; ethers such as tetrahydrofuran, dioxane, diisopropylether, diethylether, 2-methyltetrahydrofuran, cyclopentyl methyl ether, or methyl tert-butyl ether; esters such as ethyl acetate, methyl acetate, isopropyl acetate, n-propyl acetate, n-butyl acetate, isobutyl acetate, sec-butyl acetate, or tert-butyl acetate; halogenated solvents such as dichloromethane, chloroform, tetrachloromethane, dichloroethane, chlorobenzene, or dichlorobenzene; polar aprotic solvents such as N,N-dimethylformamide, acetonitrile, N,N-dimethylacetamide, N-methyl-2-pyrrolidone, or dimethylsulfoxide; hydrocarbon aliphatic solvents such as methylcyclohexane, cyclohexane, heptane, or hexane.

Preferably, the step vi) is carried out using toluene as solvent.

The step vi) can be carried out at a temperature in the range from 20° C. to 130° C., preferably from 40° C. to 115° C., more preferably at about 105° C.

The amount of methyl chloroformate (VIa-b) is from 0.8 equivalents to 10 equivalents, preferably from 1 equivalent to 5 equivalents, and more preferably from 2 equivalents to 2.5 equivalents, based on the compound of formula (VII).

The compound of formula (IX-b) can be isolated prior to the alkylation step vii), or alternatively, it can be used in the step vii) without isolation.

The compound of formula (IX-b) may be purified by any method known in the art before used in the next processing step.

The alkylation step vii) by reacting the compound of formula (IX-b) with 2,6-difluorobenzyl bromide of formula (X-a) is carried out in the presence of a base in a suitable solvent.

Non-limiting examples of suitable solvents that can be used in the alkylation step vii), which can be used alone or as a mixture of solvents, include water; aromatic hydrocarbon solvents such as toluene, benzene, o-xylene, m-xylene, p-xylene, or nitrobenzene; ketones such as acetone, methyl ethyl ketone, or methyl isobutyl ketone; ethers such as tetrahydrofuran, dioxane, diisopropylether, diethylether, 2-methyltetrahydrofuran, cyclopentyl methyl ether, or methyl tert-butyl ether; esters such as ethyl acetate, methyl acetate, isopropyl acetate, n-propyl acetate, n-butyl acetate, isobutyl acetate, sec-butyl acetate, or tert-butyl acetate; halogenated solvents such as dichloromethane, chloroform, tetrachloromethane, dichloroethane, chlorobenzene, or dichlorobenzene; polar aprotic solvents such as N,N-dimethylformamide, acetonitrile, N,N-dimethylacetamide, N-methyl-2-pyrrolidone, or dimethylsulfoxide; hydrocarbon aliphatic solvents such as methylcyclohexane, cyclohexane, heptane, or hexane.

Preferably, the step vii) is carried out using acetonitrile as solvent.

The alkylation step vii) is carried out in the presence of a base which is not particularly limited. Preferably, the base is selected from the group consisting of triethylamine, trimethylamine, diethylamine, diethanolamine, diisopropylethylamine, pyridine, dimethylaminopyridine (DMAP), dicyclohexylamine, triethanolamine, meglumine, ethylenediamine, picoline, quinoline, 1,8-diazabicyclo[5.4.0]undec-7-ene (DBU), sodium carbonate, potassium carbonate, sodium bicarbonate, potassium bicarbonate, and mixtures thereof.

Preferably, the alkylation step vii) is carried out in the presence of potassium carbonate.

The step vii) can be carried out at a temperature in the range from 20° C. to 110° C., preferably from 40° C. to 95° C. and more preferably at about 85° C.

The amount of compound (X-a) is from 0.8 equivalents to 5 equivalents, more preferably from 1 equivalent to 2.5 equivalents, and more preferably from 1.05 equivalents to 1.5 equivalents, based on the compound of formula (IX-b).

The compound of formula (XI-b) can be isolated prior to the bromination step viii), or alternatively, it can be used in the step viii) without isolation.

The compound of formula (XI-b) may be purified by any method known in the art before used in the next processing step.

The bromination step viii) is carried out in the presence of N-bromosuccinimide (NBS) and a radical initiator in a suitable solvent.

Non-limiting examples of radical initiators, which can be used alone or as a mixture of radical initiators, include 2,2′-azobis(isobutyronitrile) (AIBN), 2,2′-azobis(2,4-dimethylvaleronitrile) (AMVN), 4,4-azobis(4-cyanovaleric acid), 1,1′-azobis(cyclohexanecarbonitrile), benzoyl peroxide, and tert-butyl peroxide.

Preferably, the bromination step viii) is carried out in the presence of 2,2′-azobis(isobutyronitrile) (AIBN) as a radical initiator.

Non-limiting examples of suitable solvents that can be used in the bromination step viii), which can be used alone or as a mixture of solvents, include water; aromatic hydrocarbon solvents such as benzene or nitrobenzene; ketones such as acetone, methyl ethyl ketone, or methyl isobutyl ketone; ethers such as tetrahydrofuran, dioxane, diisopropylether, diethylether, 2-methyltetrahydrofuran, cyclopentyl methyl ether, or methyl tert-butyl ether; esters such as ethyl acetate, methyl acetate, isopropyl acetate, n-propyl acetate, n-butyl acetate, isobutyl acetate, sec-butyl acetate or tert-butyl acetate; halogenated solvents such as dichloromethane, chloroform, tetrachloromethane, dichloroethane, chlorobenzene, or dichlorobenzene; polar aprotic solvents such as N,N-dimethylformamide, acetonitrile, N,N-dimethylacetamide, N-methyl-2-pyrrolidone or dimethylsulfoxide; hydrocarbon aliphatic solvents such as methylcyclohexane, cyclohexane, heptane, or hexane.

Preferably, the step viii) is carried out using ethyl acetate as solvent.

The bromination step viii) can be carried out at a temperature in the range from 20° C. to 110° C., preferably from 40° C. to about 95° C. more preferably at about 85° C.

The bromination step viii) is carried out in the presence of N-bromosuccinimide (NBS). The amount of N-bromosuccinimide is from 0.8 equivalents to 3 equivalents, more preferably from 1 equivalent to 2.0 equivalents, and more preferably from 1.05 equivalents to 1.5 equivalents, based on the compound of formula (XI-b).

The compound of formula (XII-b) can be isolated prior to the amination step ix), or alternatively, it can be used in the step ix) without isolation.

The compound of formula (XII-b) may be purified by any method known in the art before used in the next processing step.

The amination step ix) is carried out in the presence of dimethylamine in a suitable solvent.

Non-limiting examples of suitable solvents that can be used in the amination step ix), which can be used alone or as a mixture of solvents, include water; alcohols such as methanol, ethanol, isopropanol, n-propanol, n-butanol, isobutanol, sec-butanol, or tert-butanol; aromatic hydrocarbon solvents such as benzene or nitrobenzene; ketones such as acetone, methyl ethyl ketone or methyl isobutyl ketone; ethers such as tetrahydrofuran, dioxane, diisopropylether, diethylether, 2-methyltetrahydrofuran, cyclopentyl methyl ether, or methyl tert-butyl ether; esters such as ethyl acetate, methyl acetate, isopropyl acetate, n-propyl acetate, n-butyl acetate, isobutyl acetate, sec-butyl acetate, or tert-butyl acetate; halogenated solvents such as dichloromethane, chloroform, tetrachloromethane, dichloroethane, chlorobenzene, or dichlorobenzene; polar aprotic solvents such as N,N-dimethylformamide, acetonitrile, N,N-dimethylacetamide, N-methyl-2-pyrrolidone, or dimethylsulfoxide; hydrocarbon aliphatic solvents such as methylcyclohexane, cyclohexane, heptane, or hexane.

Preferably, the step ix) is carried out using a mixture of acetone and water as solvent.

The amination step ix) can be carried out at a temperature in the range from −5° C. to 50° C., preferably from 0° C. to 25° C., and more preferably at about 5° C.

The amination step ix) is carried out in the presence of dimethylamine. The amount of dimethylamine is from 0.8 equivalents to 10 equivalents, more preferably from 1 equivalent to 7.5 equivalents, and more preferably from 3 equivalents to 5 equivalents, based on the compound of formula (XII-b).

The compound of formula (XX-b) can be isolated prior to the step x), or alternatively, it can be used in the step x) without isolation.

Preferably, the compound of formula (XX-b) formed by reacting a compound of formula (XII-b) with dimethylamine is converted in situ to the compound of formula (XX-b·oxalate).

The conversion of the compound of formula (XX-b) into the compound of formula (XX-b·oxalate) is carried out by reacting the obtained compound of formula (XX-b) with oxalic acid by any method known in the art.

In a particular embodiment, the process comprises the reaction of a compound of formula (XX-b), without isolation, with a solution of oxalic acid in an organic solvent. In another particular embodiment, the process comprises the reaction of a compound of formula (XX-b) without isolation, with oxalic acid in solid form.

Non-limiting examples of suitable solvents that can be used in the conversion of the compound of formula (XX-b) into the compound of formula (XX-b·oxalate), which can be used alone or as a mixture of solvents, include water; alcohols such as methanol, ethanol, isopropanol, n-propanol, n-butanol, isobutanol, sec-butanol or tert-butanol; aromatic hydrocarbon solvents such as benzene or nitrobenzene; ketones such as acetone, methyl ethyl ketone, or methyl isobutyl ketone; ethers such as tetrahydrofuran, dioxane, diisopropylether, diethylether, 2-methyltetrahydrofuran, cyclopentyl methyl ether, or methyl tert-butyl ether; esters such as ethyl acetate, methyl acetate, isopropyl acetate, n-propyl acetate, n-butyl acetate, isobutyl acetate, sec-butyl acetate, or tert-butyl acetate; halogenated solvents such as dichloromethane, chloroform, tetrachloromethane, dichloroethane, chlorobenzene or dichlorobenzene; polar aprotic solvents such as N,N-dimethylformamide, acetonitrile, N,N-dimethylacetamide, N-methyl-2-pyrrolidone or dimethylsulfoxide; hydrocarbon aliphatic solvents such as methylcyclohexane, cyclohexane, heptane, or hexane. Preferably, the solvent is ethanol.

The compound of formula (XX-b·oxalate) can be isolated before being used in the next processing step or can be used without isolation. Preferably, the compound of formula (XX-b·oxalate) is isolated before used in the next processing step.

The compound of formula (XX-b·oxalate) may be purified by any method known in the art before used in the next processing step.

As mentioned above, the invention also relates to intermediates used in the process disclosed herein.

As mentioned about, a second aspect of the invention relates to a compound of formula (XXVII-b·HCl).

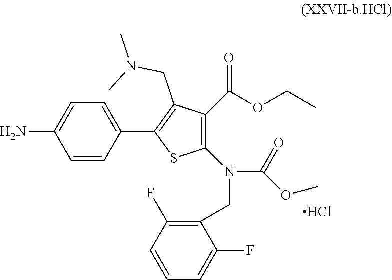 [SMILES: CCOC(=O)c1c(N(Cc2c(F)cccc2F)C(=O)OC)sc(-c2ccc(N)cc2)c1CN(C)C ; Cl]

As mentioned about, a third aspect of the invention relates to a compound of formula (XX-b·oxalate).

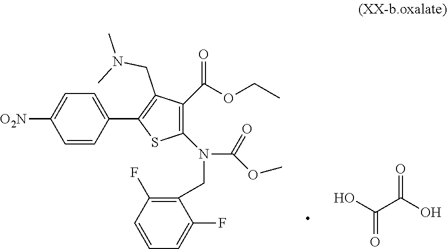 [SMILES: CCOC(=O)c1c(N(Cc2c(F)cccc2F)C(=O)OC)sc(-c2ccc([N+](=O)[O-])cc2)c1CN(C)C ; "·" ; O=C(O)C(=O)O]

### EXAMPLES

Hereinafter, the present invention is described in more detail and specifically with reference to the Examples, which however are not intended to limit the present invention.

### Example 1: Preparation of Relugolix of Formula (I)

Methyl (2,6-difluorobenzyl)(4-((dimethylamino)methyl)-3-((6-methoxypyridazin-3-yl) carbamoyl)-5-(4-(3-methoxyureido)phenyl)thiophen-2-yl)carbamate (XXVI-b) (0.5 g, 0.76 mmol) was dissolved in DMSO (1.25 ml) and 1,8-diazabicyclo(5.4.0)undec-7-ene (DBU) (0.03 g, 0.19 mmol, 0.25 eq) was added to the solution. The mixture was stirred at 20-25° C. for 24 h, ethanol (8.75 mL) was added, and the resulting mixture was stirred at 20-25° C. for 16 hours. HPLC analysis of this reaction mixture showed the presence of relugolix (97.8%), compound (XXVI-b) (0.04%), impurity A (0.07%) and impurity B (0.32%). The suspension was filtered, obtaining crude relugolix after drying at 40-50° C. under vacuum (0.37 g, 77% yield, HPLC 98.6%).

Crude relugolix samples obtained following similar procedures and having similar purities were combined (2.94 g) and were dissolved in DMSO (7.4 mL). The solution was heated up to 35° C. and filtered. Ethanol (51.5 mL) was loaded to the filtered solution, the solution was stirred at this temperature for 1 hour, then cooled down to 20-25° C. and stirred for 18 hours. The resulting suspension was filtered, washing the cake with ethanol. The obtained solid was dried at 40° C. under vacuum to obtain relugolix (2.54 g, 86.4% yield, HPLC 99.5

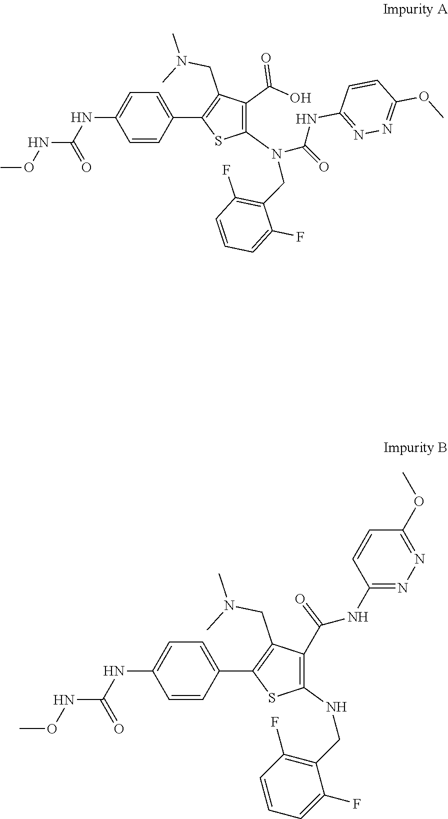

### Example 2: Preparation of Relugolix of Formula (I)

Methyl (2,6-difluorobenzyl)(4-((dimethylamino)methyl)-3-((6-methoxypyridazin-3-yl) carbamoyl)-5-(4-(3-methoxyureido)phenyl)thiophen-2-yl)carbamate (XXVI-b) (25.98 g, 0.040 mol) was dissolved in DMSO (65 mL) and 1,8-diazabicyclo(5.4.0)undec-7-ene (DBU) (1.81 g, 0.012 mmol, 0.3 eq) was added to the solution. The mixture was stirred at 20-25° C. for 24 h and heated up to 35-40° C. Then, ethanol (455 mL) was added, and during the addition, seeding with Relugolix form I was carried out. The resulting mixture was stirred at 35-40° C. for 1 hour, cooled down to 20-25° C. and stirred at this temperature for 15 hours.

The suspension was filtered, obtaining crude relugolix, which was dissolved in DMSO (56 mL) at 35-40° C. Ethanol (22.2 mL) was added and the solution was filtered, washing the filter with ethanol (56 mL). Maintaining this temperature, additional ethanol (311 mL) was loaded onto the filtered solution, and during the addition, seeding with Relugolix form I was carried out. The mixture was stirred for 1 hour, then cooled down to 20-25° C. and stirred for 15 hours. The resulting suspension was filtered, washing the cake with ethanol. The obtained solid was dried at 45-50° C. under vacuum to obtain relugolix (18.79 g, 76% yield, HPLC 99.8%).

### Example 3: Preparation of ethyl 2-((2,6-difluorobenzyl)(methoxycarbonyl)amino)-4-((dimethylamino)methyl)-5-(4-(3-methoxyureido)phenyl)thiophene-3-carboxylate of formula (XXVIII-b)

A mixture of 1,1′-carbonyldiimidazole (CDI) (20.42 g, 0.126 mol, 1.7 eq), acetonitrile (120 mL) and triethylamine (6.37 g, 0.063 mol, 0.85 eq) was cooled down to 5-10° C. Methoxyamine hydrochloride (11.75 g, 0.141 mol, 1.9 eq) was added in portions keeping this temperature. The suspension was warmed to 20-25° C. and sodium carbonate (8.64 g, 0.081 mol, 1.1 eq) was added. Then, ethyl 5-(4-aminophenyl)-2-((2,6-difluorobenzyl)(methoxycarbonyl)amino)-4-((dimethylamino)methyl)thiophene-3-carboxylate hydrochloride (XXVII-b·HCl) (40 g, 0.074 mol) was added. The mixture was heated up to 48-52° C. and stirred at this temperature for 2 hours. The reaction was cooled down to 40° C., deionized water (160 mL) was added, and the mixture was cooled down to 20-25° C. Deionized water (160 mL) was added onto the resulting suspension, and then the mixture was cooled down to 0-5° C. The resulting suspension was stirred for 1 hour and filtered, and the cake was washed with deionized water. The obtained solid was dried at 40-45° C. under vacuum until constant weight to obtain the compound of formula (XXVIII-b) (34 g, 80% yield, H PLC 98.8%).

### Example 4: Preparation of ethyl 2-((2,6-difluorobenzyl)(methoxycarbonyl)amino)-4-((dimethylamino)methyl)-5-(4-(3-methoxyureido)phenyl)thiophene-3-carboxylate of formula (XXVIII-b)

A mixture of 1,1′-carbonyldiimidazole (CDI) (76.57 g, 0.472 mol, 1.7 eq), acetonitrile (450 mL) and triethylamine (23.89 g, 0.236 mol, 0.85 eq) was cooled down to 5-10° C.

Methoxyamine hydrochloride (44.08 g, 0.528 mol, 1.9 eq) was added in portions. The mixture was warmed to 20-25° C. and stirred until complete dissolution. Then, it was loaded onto a flask containing ethyl 5-(4-aminophenyl)-2-((2,6-difluorobenzyl)(methoxycarbonyl)amino)-4-((dimethylamino)methyl)thiophene-3-carboxylate hydrochloride (XXVII-b·HCl) (150 g, 0.278 mol). Finally, sodium carbonate (32.38 g, 0.306 mol, 1.1 eq) was added. The mixture was heated up to 45-50° C. and stirred at this temperature for 2 hours. The reaction was cooled down to 40° C., deionized water (600 mL) was added, the mixture was cooled down to 20-25° C., seeded with solid compound of formula (XXVIII-b) and stirred for about 30 minutes. Deionized water (600 mL) was added, and then the mixture was cooled down to 0-5° C. and stirred for 1 hour. The suspension was filtered, and the cake was washed with deionized water. The obtained wet solid corresponded to the compound of formula (XXVIII-b) (123.1 g dry equivalent, 77% yield, HPLC 98.7%).

### Example 5: Preparation of 2-((2,6-difluorobenzyl)(methoxycarbonyl)amino)-4-((dimethylamino)methyl)-5-(4-(3-methoxyureido)phenyl)thiophene-3-carboxylic acid of formula (XXIX-b)

Ethyl 2-((2,6-difluorobenzyl)(methoxycarbonyl)amino)-4-((dimethylamino)methyl)-5-(4-(3-methoxyureido)phenyl)thiophene-3-carboxylate (XXVIII-b) (30 g, 0.052 mol) was dissolved in acetone (90 mL) at 20-25° C. then deionized water (120 mL) was added together with 50% w/w aqueous NaOH (5.41 g, 0.067 mol, 1.3 eq). The resulting solution was heated up to 45-50° C. and stirred for 26 h at this temperature. The reaction mixture was cooled down to 20-25° C. and pH was adjusted to 6.0-7.0 with concentrated HCl. The mixture was concentrated to dryness under reduced pressure. Isopropanol (86 mL) was added onto the residue and the resulting suspension was concentrated to dryness under reduced pressure.

Isopropanol (71 mL) was added onto the residue, the mixture was heated up to 60° C. and stirred for 10 min. The suspension was cooled down to 20-25° C. and was filtered. The cake was washed with isopropanol. The resulting solid was suspended in acetonitrile (274 mL) and the suspension was heated up to 60-70° C. Deionized water (23 mL) was added, and the light suspension was filtered washing the filter with acetonitrile. The filtered solution was distilled under vacuum to a final residue of approximately 170 mL. The suspension was cooled down to 0-(−5) ° C., stirred for 1 hour and filtered, and the obtained cake was washed with cold acetonitrile. The obtained solid was dried at 45-50° C. under vacuum to obtain the compound of formula (XXIX-b) (16.65 g, 58% yield, HPLC 99.4%).

### Example 6: Preparation of 2-((2,6-difluorobenzyl)(methoxycarbonyl)amino)-4-((dimethylamino)methyl)-5-(4-(3-methoxyureido)phenyl)thiophene-3-carboxylic acid of formula (XXIX-b)

Ethyl 2-((2,6-difluorobenzyl)(methoxycarbonyl)amino)-4-((dimethylamino)methyl)-5-(4-(3-methoxyureido)phenyl)thiophene-3-carboxylate (XXVIII-b) (100 g dry equivalent, 0.173 mol) was dissolved in acetone (300 mL) at 20-25° C., then deionized water (400 mL) was added together with 50% w/w aqueous NaOH (18.04 g, 0.225 mol, 1.3 eq). The resulting solution was heated up to 45-50° C. and stirred for 26 h at this temperature. The reaction mixture was cooled down to 20-25° C. and pH was adjusted to 6.0-7.0 with concentrated HCl. The mixture was concentrated to dryness under reduced pressure. Isopropanol (285 mL) was added onto the residue and the resulting suspension was concentrated to dryness under reduced pressure. Isopropanol (238 mL) was added onto the residue, the mixture was heated up to 60° C. and stirred for 10 min. The suspension was cooled down to 20-25° C. and was filtered. The cake was washed with isopropanol. The resulting solid was suspended in acetonitrile (913 mL) and the suspension was heated up to 65-70° C. Deionized water (76 mL) was added, and the light suspension was filtered washing the filter with acetonitrile. The filtered solution was distilled under vacuum to a final residue of approximately 913 mL. Acetonitrile (304 mL) was added and the mixture was distilled under vacuum to a final residue of approximately 913 mL. Acetonitrile (304 mL) was added and the mixture was distilled under vacuum to a final residue of approximately 571 mL. The suspension was cooled down to 0-(−5) ° C., stirred for 1 hour and filtered, and the obtained cake was washed with cold acetonitrile. The obtained solid was dried at 45-50° C. under vacuum to obtain the compound of formula (XXIX-b) (88.0 g, 93% yield, HPLC 99.1%).

### Example 7: Preparation of methyl (2,6-difluorobenzyl)(4-((dimethylamino)methyl)-3-((6-methoxypyridazin-3-yl)carbamoyl)-5-(4-(3-methoxyureido)phenyl)thiophen-2-yl)carbamate of formula (XXVI-b)

2-((2,6-Difluorobenzyl)(methoxycarbonyl)amino)-4-((dimethylamino)methyl)-5-(4-(3-methoxyureido)phenyl)thiophene-3-carboxylic acid (XXIX-b) (15.0 g, 0.027 mol) was mixed with ethyl acetate (150 mL) and diisopropylethylamine (9.54 g, 0.074 mol, 2.7 eq) at 20-25° C. The mixture was stirred until dissolution. Afterwards, 6-methoxypyridazin-3-amine (XVIII) (5.82 g, 0.046 mol, 1.7 eq) and propylphosphonic anhydride (T3P) 50% w/w in ethyl acetate solution (43.5 g, 0.068 mol, 2.5 eq) were added. The mixture was heated up to 35-40° C. and stirred at this temperature for 4 hours. The reaction was cooled down to 10-15° C., deionized water (75 mL) and ethyl acetate (150 mL) were added, followed by the addition of Na₂CO₃ until the pH of the aqueous phase was 8-9. The biphasic mixture was stirred for 15 minutes, and the organic phase was separated. The organic phase was distilled under atmospheric pressure until a final residue of approximately 50 mL. The suspension was cooled down to 0-(−5) ° C., stirred for 1 hour and filtered. The cake was washed with cold ethyl acetate. The obtained solid was dried at 40-45° C. under vacuum to obtain the compound of formula (XXVI-b) (14.9 g, 83% yield, HPLC 97.7%).

### Example 8: Preparation of methyl (2,6-difluorobenzyl)(4-((dimethylamino)methyl)-3-((6-methoxypyridazin-3-yl)carbamoyl)-5-(4-(3-methoxyureido)phenyl)thiophen-2-yl)carbamate of formula (XXVI-b)

2-((2,6-Difluorobenzyl)(methoxycarbonyl)amino)-4-((dimethylamino)methyl)-5-(4-(3-methoxyureido)phenyl)thiophene-3-carboxylic acid (XXIX-b) (30.0 g, 0.055 mol) was mixed with 6-methoxypyridazin-3-amine (XVIII) (8.21 g, 0.066 mol, 1.2 eq), diisopropylethylamine (12.72 g, 0.098 mol, 1.8 eq) and ethyl acetate (240 mL) and at 20-25° C. The mixture was stirred for 10 minutes. Afterwards, propylphosphonic anhydride (T3P) 50% w/w in ethyl acetate solution (55.7 g, 0.088 mol, 1.6 eq) was added in 10-15 minutes keeping the temperature below 35° C. The mixture was heated up to 45-50° C. and stirred at this temperature for 1 hour. The reaction was cooled down to 20-25° C. and it was submitted to distillation under vacuum until a final residue of approximately 135 mL. The distillation residue was cooled down to 5-10° C. and it was slowly added deionized water (175 mL) maintaining the temperature below 15° C. The mixture was warmed to 15-20° C. and the pH was adjusted to 8-8.5 with a solution of Na₂CO₃ (5 g) in deionized water (820 mL). The resulting suspension was cooled down to 5-10° C., stirred for 1 hour and filtered. The cake was washed with cold deionized water and then with cold ethyl acetate.

The wet solid was treated with DMSO (81 mL), and the mixture was heated up to 35-40° C. Ethanol (485 mL) was added, keeping this temperature. The suspension was stirred at this temperature for 1 hour, cooled down to 20-25° C. and further stirred for 15 hours, cooled down again to 10° C. and stirred for 1 hour. The suspension was filtered, washing the filter with cold ethanol.

The obtained wet solid corresponded to the compound of formula (XXVI-b) (26.96 g dry equivalent, 75% yield, HPLC 99.5%).

### Example 9: Preparation of ethyl 2-((2,6-difluorobenzyl)(methoxycarbonyl)amino)-4-((dimethylamino) methyl)-5-(4-nitrophenyl)thiophene-3-carboxylate of formula (XX-b)

Ethyl 2-((2,6-difluorobenzyl)(methoxycarbonyl)amino)-4-((dimethylamino) methyl)-5-(4-nitrophenyl)thiophene-3-carboxylate oxalate (XX-b oxalate) (14.81 g) and potassium carbonate (5.25 g) were suspended in ethyl acetate (74 mL), the temperature was lowered to 0-10° C., and deionized water (74 mL) was loaded keeping this temperature. The mixture was stirred for about 1 hour at 0-10° C., then it was warmed to 20-25° C. and the phases were allowed to settle. The aqueous phase was separated, and the organic phase was washed twice with deionized water (2×30 mL), distilled at atmospheric pressure until a final residual volume of approximately 25 mL. Ethyl acetate (30 mL) was loaded, and distillation was repeated until the same residual volume. Again, ethyl acetate (30 mL) was loaded, and distillation was repeated until the same residual volume. The volume of the final solution was adjusted to approximately 185 mL and was used as such. The solution contains approximately 12.6 g of compound of formula (XX-b).

### Example 10: Preparation of ethyl 2-((2,6-difluorobenzyl)(methoxycarbonyl)amino)-4-((dimethylamino) methyl)-5-(4-nitrophenyl)thiophene-3-carboxylate of formula (XX-b)

Potassium carbonate (11.97 g) was dissolved in deionized water (150 mL) and the solution was cooled down to 5-10° C. Ethyl 2-((2,6-difluorobenzyl)(methoxycarbonyl)amino)-4-((dimethylamino) methyl)-5-(4-nitrophenyl)thiophene-3-carboxylate oxalate (XX-b oxalate) (30 g dry equivalent, 0.048 mol) was added followed by the load of ethyl acetate (150 mL). The mixture was stirred at 5-10° C. for 1 hour, warmed to 20-25° C. and stirred for 2 additional hours. The phases were allowed to settle and the pH of the aqueous phase was checked to be 8-8.5. The aqueous phase was separated, and the organic phase was washed twice with deionized water (2×60 mL), distilled at atmospheric pressure until a final residual volume of approximately 45 mL. Ethyl acetate (60 mL) was loaded, and distillation was repeated until the same residual volume. Again, ethyl acetate (60 mL) was loaded, and distillation was repeated until the same residual volume. Ethyl acetate (103 mL) and active charcoal (1.3 g) were added onto the distillation residue, the mixture was stirred at 20-25° C. for 1 h and it was filtered, washing the filter with ethyl acetate (13 mL). Active charcoal (1.3 g) was added onto the filtered solution, the mixture was stirred at 20-25° C. for 1 hour and it was filtered, washing the filter with ethyl acetate (13 mL).

The volume of the final solution was adjusted to approximately 370 mL with ethyl acetate and was used as such. The solution contains approximately 25.67 g of compound of formula (XX-b).

### Example 11: Preparation of ethyl 5-(4-aminophenyl)-2-((2,6-difluorobenzyl)(methoxycarbonyl)amino)-4-((dimethylamino)methyl)thiophene-3-carboxylate hydrochloride of formula (XXVII-b·HCl)

To a solution containing ethyl 2-((2,6-difluorobenzyl)(methoxycarbonyl)amino)-4-((dimethylamino)methyl)-5-(4-nitrophenyl)thiophene-3-carboxylate (XX-b) (1.5 g, 3.0 mmol) in ethyl acetate (21 mL), it was loaded wet 10% Pd/C (0.03 g dry equivalent, 2% w/w) and the mixture was stirred at 25-30° C. for 4 h under a hydrogen pressure of 2 bar. The catalyst was removed by filtration and the filtered solution was concentrated under atmospheric pressure until a final residue of about 15 mL. 20% w/w solution of hydrogen chloride in isopropanol (0.56 g, 3.1 mmol, 1.1 eq) was added at 20-25° C. The resulting suspension was stirred at 20-25° C. for 1 h and filtered. The cake was washed with ethyl acetate. The solid was dried at 40-45° C. under vacuum until constant weight to obtain the compound of formula (XXVII-b·HCl) (1.23 g, 81% yield, HPLC 99.5%).

### Example 12: Preparation of ethyl 5-(4-aminophenyl)-2-((2,6-difluorobenzyl)(methoxycarbonyl)amino)-4-((dimethylamino)methyl)thiophene-3-carboxylate hydrochloride of formula (XXVII-b·HCl)

To a solution containing ethyl 2-((2,6-difluorobenzyl)(methoxycarbonyl)amino)-4-((dimethylamino)methyl)-5-(4-nitrophenyl)thiophene-3-carboxylate (XX-b) (25.67 g, 0.048 mol) in ethyl acetate (approximately 355 mL), it was loaded wet 10% Pd/C (0.51 g dry equivalent, 2% w/w) and the mixture was stirred at 25-30° C. for 4 h under a hydrogen pressure of 2 bar. The catalyst was removed by filtration and the filtered solution was concentrated under atmospheric pressure until a final residue of about 208 mL. 18.56% w/w solution of hydrogen chloride in isopropanol (8.52 g) was added at 20-25° C. The resulting suspension was stirred at 20-25° C. for 1 h and filtered. The cake was washed with ethyl acetate. The solid was dried at 40-45° C. under vacuum until constant weight to obtain the compound of formula (XXVII-b·HCl) (19.97 g, 77% yield, HPLC 99.5%).

### Example 13: Preparation of ethyl 2-((methoxycarbonyl)amino)-4-methyl-5-(4-nitrophenyl)thiophene-3-carboxylate of formula (IX-b)

Ethyl 2-amino-4-methyl-5-(4-nitrophenyl)thiophene-3-carboxylate (VII) (100 g, 0.326 mol) 5 was suspended in toluene (250 mL). The suspension was heated up to 98-105° C. Methyl chloroformate (VIII-b) (67.9 g, 0.718 mol, 2.2 eq) was added dropwise for 2 hours. The addition funnel was washed with toluene (50 mL). The mixture was stirred at this temperature for 3 hours. The mixture was cooled down to 60-70° C. and methanol (900 mL) was added dropwise. The mixture was heated up to reflux temperature and stirred for 3 hours. The resulting suspension was cooled down to 20-25° C., stirred at this temperature for 1 hour and filtered. The solid obtained was washed with methanol and then with heptane. The collected solid, corresponding to the compound of formula (IX-b), was dried at 45-50° C. under vacuum until constant weight (116.5 g, 98% yield, HPLC 99.9%, yellow solid).

### Example 14: Preparation of ethyl 2-((methoxycarbonyl)amino)-4-methyl-5-(4-nitrophenyl)thiophene-3-carboxylate of formula (IX-b)

Ethyl 2-amino-4-methyl-5-(4-nitrophenyl)thiophene-3-carboxylate (VII) (103 g, 0.336 mol) was suspended in toluene (282 mL). The suspension was heated up to 95-105° C. Methyl chloroformate (VIII-b) (70 g, 0.741 mol, 2.2 eq) was added dropwise for 2 hours. The addition funnel was washed with toluene (11.5 mL). The mixture was stirred at this temperature for 5 hours. The mixture was cooled down to 60-70° C. and methanol (866 mL) was added dropwise. The mixture was stirred for 3 hours at this temperature. The resulting suspension was cooled down to 20-25° C., stirred at this temperature for 1 hour and filtered. The solid obtained was washed with methanol and then with heptane. The solid, corresponding to the compound of formula (IX-b), was dried at 45-55° C. under vacuum until constant weight (112.7 g, 92% yield, HPLC 100.0%, yellow solid).

### Example 15: Preparation of ethyl 2-((2,6-difluorobenzyl)(methoxycarbonyl)amino)-4-methyl-5-(4-nitrophenyl)thiophene-3-carboxylate of formula (XI-b)

Ethyl 2-((methoxycarbonyl)amino)-4-methyl-5-(4-nitrophenyl)thiophene-3-carboxylate (IX-b) (75.17 g, 0.206 mol), 2,6-difluorobenzyl bromide (X-a) (47.2 g, 0.227 mol, 1.1 eq) and potassium carbonate (31.34 g, 0.227 mol, 1.1 eq) were mixed with acetonitrile (376 mL), the suspension was heated up to reflux and stirred at this temperature for 10 hours. The reaction mixture was cooled down to 45-55° C. and it was distilled under vacuum until a remaining residue of approximately 90 mL. Deionised water (376 mL) was added at 45-55° C. followed by the slow addition of heptane (451 mL). The resulting suspension was cooled down to 0-5° C. stirred at this temperature for 1 hour and filtered. The solid was washed successively with cold deionized water (368 mL), cold heptane (180 mL) and three times with a mixture 3:1 w/w of heptane/ethyl acetate (75.2 mL). The collected solid, corresponding to the compound of formula (XI-b), was dried at 50° C. under vacuum (81.79 g, 81% yield, HPLC 99.9%).

### Example 16: Preparation of ethyl 2-((2,6-difluorobenzyl)(methoxycarbonyl)amino)-4-methyl-5-(4-nitrophenyl)thiophene-3-carboxylate of formula (XI-b)

Ethyl 2-((methoxycarbonyl)amino)-4-methyl-5-(4-nitrophenyl)thiophene-3-carboxylate (IX-b) (90.0 g, 0.247 mol), 2,6-difluorobenzyl bromide (X-a) (56.5 g, 0.273 mol, 1.1 eq) and potassium carbonate (37.5 g, 0.273 mol, 1.1 eq) were mixed with acetonitrile (453 mL), the suspension was heated up to reflux and stirred at this temperature for 10 hours. The reaction mixture was cooled down to 50-55° C. and it was distilled under vacuum until a remaining residue of approximately 200 mL. Deionized water (454 mL) was added at 50-55° C. followed by the slow addition of heptane (539 mL). The resulting suspension was cooled down to 0-5° C., stirred at this temperature for 1 hour and filtered. The solid was washed successively with cold deionized water (100 mL), cold heptane (146 mL) and three times with a mixture 3:1 w/w of heptane/ethyl acetate (3×47 mL). The collected solid, corresponding to the compound of formula (XI-b), was dried at 50° C. under vacuum (118.2 g, 97.5% yield, HPLC 100.0%).

### Example 17: Preparation of ethyl 4-(bromomethyl)-2-((2,6-difluorobenzyl)(methoxycarbonyl)amino)-5-(4-nitrophenyl)thiophene-3-carboxylate of formula (XII-b)

Ethyl 2-((2,6-difluorobenzyl)(methoxycarbonyl)amino)-4-methyl-5-(4-nitrophenyl) thiophene-3-carboxylate (XI-b) (99.98 g, 0.204 mol), 2,2′-azobis(isobutyronitrile) (AIBN, 10.03 g, 0.061 mol, 0.3 eq) and N-bromosuccinimide (NBS, 54.51 g, 0.306 mol, 1.5 eq) were mixed with ethyl acetate (500 mL) and deionized water (3.93 mL). The suspension was heated up to reflux temperature and stirred at this temperature for 1 hour. The reaction mixture was cooled down to 20-25° C. and deionized water (400 mL) was loaded together with additional ethyl acetate (100 mL) and brine (55 mL). The aqueous phase was separated. The organic phase was washed with deionized water (200 mL) and brine (55 mL) and concentrated under reduced pressure until an oily mass. Ethyl acetate (100 ml) was added at 50-55° C., the mixture was cooled down to 20-25° C. and heptane (400 mL) was added slowly for at least 1 hour. The suspension was stirred for 2 hours at this temperature, then it was cooled down to 0-5° C., stirred for 2 hours at this temperature and filtered. The cake was washed three times with a mixture 2:1 v/v of heptane/ethanol (3×150 mL). The collected solid corresponds to the compound of formula (XII-b) (98.2 g dry equivalent, 85% yield, HPLC 94.3%).

### Example 18: Preparation of ethyl 4-(bromomethyl)-2-((2,6-difluorobenzyl)(methoxycarbonyl)amino)-5-(4-nitrophenyl)thiophene-3-carboxylate of formula (XII-b)

Ethyl 2-((2,6-difluorobenzyl)(methoxycarbonyl)amino)-4-methyl-5-(4-nitrophenyl) thiophene-3-carboxylate (XI-b) (118 g, 0.241 mol), 2,2′-azobis(isobutyronitrile) (AIBN, 11.8 g, 0.072 mol, 0.3 eq) and N-bromosuccinimide (NBS, 64.3 g, 0.361 mol, 1.5 eq) were mixed with ethyl acetate (589 mL) and deionized water (4.72 mL). The suspension was heated up to reflux temperature and stirred at this temperature for 1 hour. The reaction mixture was cooled down to 20-30° C. and deionized water (472 mL) was loaded together with additional ethyl acetate (118 mL) and brine (83 mL). After stirring for 15 minutes and allowing the phases to settle, the aqueous phase was separated. The organic phase was washed with deionized water (236 mL) and brine (83 mL) and concentrated under reduced pressure until an oily mass. The mixture was flushed under reduced pressure two times with ethyl acetate (2×118 mL). Ethyl acetate (118 mL) was added at 50-55° C., the mixture was cooled down to 20-30° C. and heptane (471 mL) was added slowly for at least 1 hour. The suspension was stirred for 2 hours at this temperature, then it was cooled down to 0-5° C., stirred for 2 hours at this temperature and filtered. The cake was washed three times with a mixture 2:1 v/v of heptane/ethanol (3×70 mL). The collected solid corresponds to the compound of formula (XII-b) (118.3 g dry equivalent, 86% yield, HPLC 96.5%).

### Example 19: Preparation of ethyl 2-((2,6-difluorobenzyl)(methoxycarbonyl)amino)-4-((dimethylamino) methyl)-5-(4-nitrophenyl)thiophene-3-carboxylate oxalate (XX-b·oxalate)

Dimethylamine 40% in water (78 mL, 0.615 mol, 5 eq) was dissolved in acetone (280 mL) at 20-30° C. The solution was cooled down to 0-10° C. and a suspension of ethyl 4-(bromomethyl)-2-((2,6-difluorobenzyl)(methoxycarbonyl)amino)-5-(4-nitrophenyl) thiophene-3-carboxylate (XII-b) (69.98 g dry equivalent, 0.123 mol) in acetone (350 mL) was slowly added at this temperature. The dropping funnel was washed with acetone (70 mL). The mixture was stirred at 0-10° C. for 1 h. The resulting suspension was heated up to 30-40° C. and distilled under vacuum at a temperature below 40° C. until a final residue of approximately 110 mL. The residue was cooled down to 20-25° C. and ethyl acetate (350 mL) and an aqueous solution of Na₂CO₃ (13 g in 350 mL) were added. The biphasic mixture was stirred, and the aqueous phase was separated. The organic phase was washed with an aqueous solution of Na₂CO₃ (13 g in 350 mL) and then with a mixture of deionised water (175 mL) and brine (39 mL). The organic phase was distilled under reduced pressure until a final residue of approximately 110 mL. Ethyl acetate (175 mL) was added, and the resulting mixture was distilled under vacuum until a final residue of 110 mL. Ethyl acetate (175 mL) was added, and the resulting mixture was distilled under vacuum until a final residue of 110 mL. Ethyl acetate (280 mL) was added followed by active charcoal (3.5 g); the dark mixture was stirred at 20-25° C. for 1 hour and filtered, washing the filter with ethyl acetate (2×140 mL). A mixture of oxalic acid dihydrate (23.24 g) in ethanol (35 mL) was dissolved by heating and the solution was slowly added onto the previous filtered organic solution, rinsing the material with ethyl acetate (70 mL). The suspension was stirred for 4 hours at 20-25° C., cooled down to 0-5° C. and stirred for 2 hours. The resulting suspension was filtered, and the cake was washed with ethyl acetate to obtain the compound of formula (XX-b·oxalate) (48 g dry equivalent, 63%, HPLC 99.6%).

### Example 20: Preparation of ethyl 2-((2,6-difluorobenzyl)(methoxycarbonyl)amino)-4-((dimethylamino) methyl)-5-(4-nitrophenyl)thiophene-3-carboxylate oxalate (XX-b·oxalate)

Dimethylamine 40% in water (117 g, 1.036 mol, 5 eq) was dissolved in acetone (470 mL) at 20-30° C. The solution was cooled down to 0-10° C. and a suspension of ethyl 4-(bromomethyl)-2-((2,6-difluorobenzyl)(methoxycarbonyl)amino)-5-(4-nitrophenyl) thiophene-3-carboxylate (XII-b) (118 g dry equivalent, 0.207 mol) in acetone (588 mL) was slowly added at this temperature. The mixture was stirred at 0-10° C. for 1 h. The resulting suspension was heated up to 30-40° C. and distilled under vacuum at a temperature below 40° C. until almost to dryness. The residue was cooled down to 20-30° C. and ethyl acetate (589 mL) and an aqueous solution of Na₂CO₃ (11 g in 295 mL) were added. The biphasic mixture was stirred, and the aqueous phase was separated. The organic phase was washed with an aqueous solution of Na₂CO₃ (11 g in 295 mL) and then with a mixture of deionised water (380 mL) and sodium chloride (33 g). The organic phase was distilled under reduced pressure until almost to dryness. The mixture was flushed under reduced pressure two times with ethyl acetate (2×295 mL). Ethyl acetate (472 mL) was added followed by active charcoal (6 g); the dark mixture was stirred at 20-30° C. for 1 hour and filtered, washing the filter with ethyl acetate (2×235 mL). Oxalic acid dihydrate (39 g) was added onto the previous filtered organic solution. The suspension was stirred for 4 hours at 20-30° C., cooled down to 0-5° C. and stirred for 2 hours. The resulting suspension was filtered, and the cake was washed with ethyl acetate to obtain the compound of formula (XX-b·oxalate) (124 g dry equivalent, 93%, HPLC 98.9%).

### Comparative Example 1: Preparation of Relugolix

Methyl (2,6-difluorobenzyl)(4-((dimethylamino)methyl)-3-((6-methoxypyridazin-3-yl) carbamoyl)-5-(4-(3-methoxyureido)phenyl)thiophen-2-yl)carbamate (XXVI-b) (0.5 g, 0.76 mmol) was mixed with methanol (4.8 mL) and 30% methanolic solution of sodium methoxide (0.28 mL, 1.53 mmol, 2 eq), and the mixture was stirred at 20-25° C. for 6 hours. Then, additional sodium methoxide (0.07 mL, 0.38 mmol, 0.5 eq) was loaded and the mixture was stirred for 18 h. The solvent was distilled off and the HPLC analysis of the reaction mixture residue showed the presence of relugolix (6.6%), compound XXVI-b (3.4%), impurity A (9.9%) and impurity B (61.8%).

### Comparative Example 2: Preparation of Relugolix

Methyl (2,6-difluorobenzyl)(4-((dimethylamino)methyl)-3-((6-methoxypyridazin-3-yl) carbamoyl)-5-(4-(3-methoxyureido)phenyl)thiophen-2-yl)carbamate (XXVI-b) (0.6 g, 0.92 mmol) was mixed with methanol (6 mL) and sodium ethoxide (0.12 g, 1.83 mmol, 2.0 eq), and the mixture was stirred at 20-25° C. for 2 hours. HPLC analysis of this reaction mixture showed the presence of relugolix (48.0%), compound XXVI-b (0.2%), impurity A (10.2%) and impurity B (39.6%).

### Comparative Example 3: Preparation of Relugolix

Methyl (2,6-difluorobenzyl)(4-((dimethylamino)methyl)-3-((6-methoxypyridazin-3-yl) carbamoyl)-5-(4-(3-methoxyureido)phenyl)thiophen-2-yl)carbamate (XXVI-b) (0.5 g, 0.76 mmol) was mixed with methanol (6 mL) and sodium t-butoxide (0.15 g, 1.53 mmol, 2.0 eq), and the mixture was stirred at 20-25° C. for 2 hours. HPLC analysis of this reaction mixture showed the presence of Relugolix (78.0%), compound XXVI-b (2.4%), impurity A (3.7%) and impurity B (14.6%).

Table 1 below summarizes the HPLC results as measured in samples from the reaction mixture obtained after the cyclization step of the compound of formula (XXVI-b) using different bases.

TABLE 1

| | | | Compound | Impurity | Impurity |
|---|---|---|---|---|---|
| | Base | Relugolix | (XXVI-b) | A | B |
| Example 1 | DBU | 97.8% | 0.04% | 0.07% | 0.32% |
| Comparative | NaOMe | 6.6% | 3.4% | 9.9% | 61.8% |
| Example 1 | | | | | |
| Comparative | NaOEt | 48.0% | 0.2% | 10.2% | 39.6% |
| Example 2 | | | | | |
| Comparative | NaO^{t}Bu | 78.0% | 2.4% | 3.7% | 14.6% |
| Example 3 | | | | | |

What is claimed is:

1. A process for the preparation of relugolix of formula (I), or a pharmaceutically acceptable salt thereof, which comprises the steps of:
a) cyclizing a compound of formula (XXVI-b) in the presence of a base selected from the group consisting of 1,5,7-triazabicyclo[4.4.0]dec-5-ene (TBD), 7-methyl-1,5,7-triazabicyclo[4.4.0]dec-5-ene (MTBD), 1,8-diazabicyclo[5.4.0]undec-7-ene (DBU), 1,5-diazabicyclo[4.3.0]non-5-ene (DBN), and 1,1,3,3-tetramethylguanidine (TMG) in the presence of a suitable solvent to obtain relugolix, and 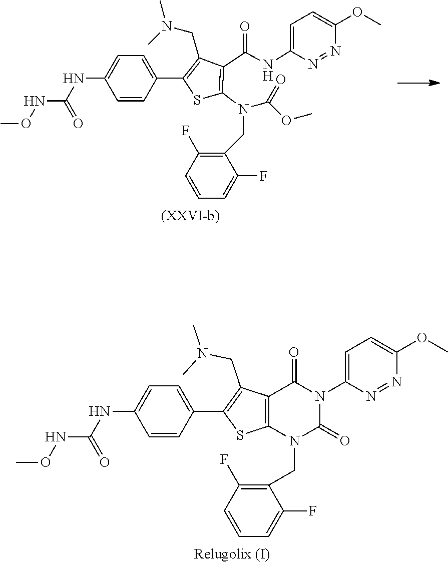
b) optionally converting the relugolix of formula (I) into a pharmaceutically acceptable salt thereof.

2. The process according to claim 1, wherein the base is 1,8-diazabicyclo[5.4.0]undec-7-ene (DBU).

3. The process according to claim 1, which previous to step a), further comprises the steps of:
i) coupling a compound of formula (XXVII-b·HCl) with methoxyamine or a salt thereof in the presence of a coupling agent and a suitable solvent to give a compound of formula (XXVIII-b), 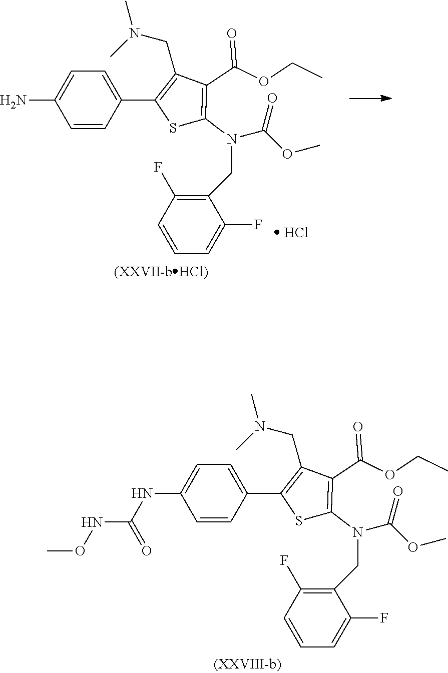
ii) hydrolyzing the compound of formula (XXVIII-b) obtained in step (i) to give a compound of formula (XXIX-b), and 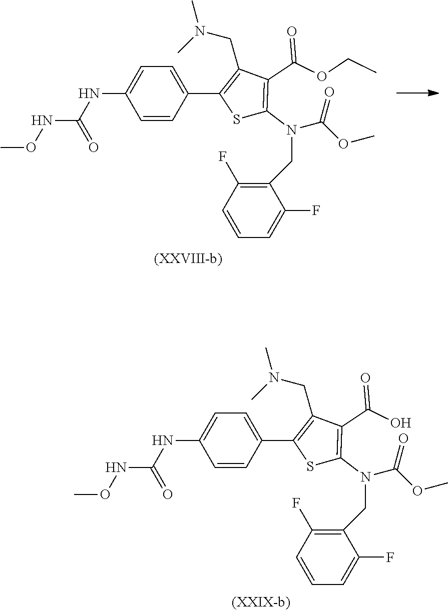
iii) coupling the compound of formula (XXIX-b) obtained in step (ii) with 3-amino-6-methoxypyridazine of formula (XVIII) in the presence of a coupling agent and in a suitable solvent to give a compound of formula (XXVI-b).

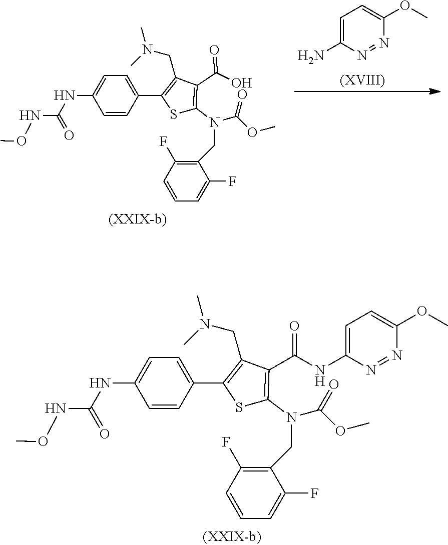

4. The process according to claim 3, wherein the methoxyamine is in the form of its hydrochloride salt.

5. The process according to claim 3, wherein the coupling agent in step i) is 1,1′-carbonyldiimidazole (CDI).

6. The process according to claim 3, wherein the hydrolysis agent in step ii) is sodium hydroxide.

7. The process according to claim 3, wherein the coupling agent in step iii) is propylphosphonic anhydride (T3P).

8. The process according to claim 3, which previous to step i), further comprises the steps of:
iv) neutralizing a compound of formula (XX-b·oxalate) to obtain a compound of formula (XX-b), and 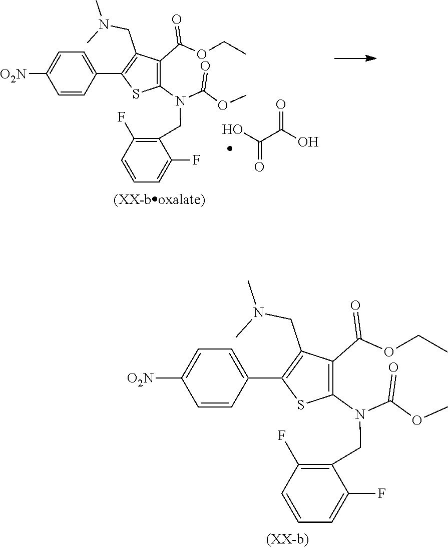
v) reducing the compound of formula (XX-b) obtained in step (iv) to obtain a compound of formula (XXVII-b), which is further converted to a compound of formula (XXVII-b·HCl).

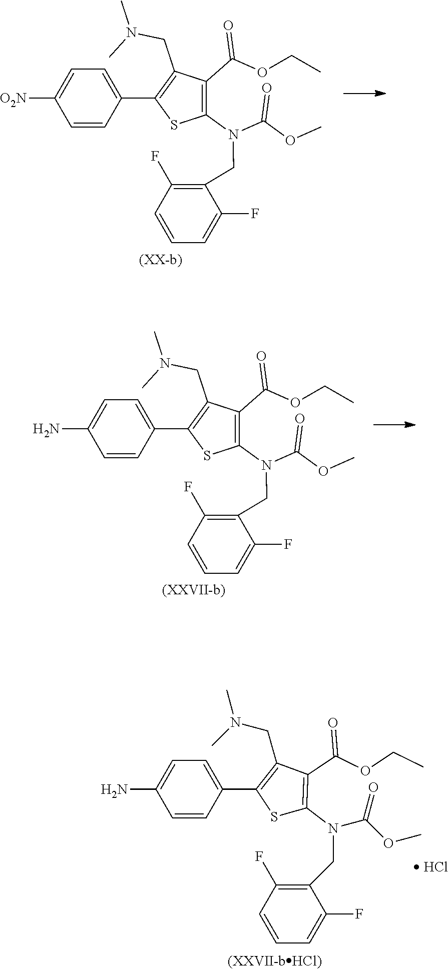 [SMILES: CCOC(=O)c1c(N(Cc2c(F)cccc2F)C(=O)OC)sc(-c2ccc([N+](=O)[O-])cc2)c1CN(C)C ; CCOC(=O)c1c(N(Cc2c(F)cccc2F)C(=O)OC)sc(-c2ccc(N)cc2)c1CN(C)C ; CCOC(=O)c1c(N(Cc2c(F)cccc2F)C(=O)OC)sc(-c2ccc(N)cc2)c1CN(C)C ; "· HCl"]

9. The process according to claim 8, wherein the neutralization agent in step iv) is potassium carbonate.

10. The process according to claim 8, wherein the reduction step v) comprises the catalytic hydrogenation of a compound of formula (XX-b) with hydrogen in the presence of a catalyst.

11. The process according to claim 8, wherein the conversion of the compound of formula (XXVII-b) into a compound of formula (XXVII-b·HCl) comprises reacting the compound of formula (XXVII-b) with hydrogen chloride (HCl).

12. The process according to claim 8, which previously to step iv), further comprises the steps of:
vi) reacting a compound of formula (VII) with methyl chloroformate of formula (Vill-b) to give a compound of formula (IX-b),
vii) reacting the compound of formula (IX-b) obtained in step (vi) with 2,6-difluorobenzyl bromide of formula (X-a) to give a compound of formula (XI-b),
viii) brominating the compound of formula (XI-b) obtained in step (vii) with N-bromosuccinimide (NBS) to obtain a compound of formula (XII-b),
ix) reacting the compound of formula (XII-b) obtained in step (viii) with dimethylamine to give a compound of formula (XX-b), and
x) reacting the compound of formula (XX-b) obtained in step ix) with oxalic acid to obtain a compound of formula (XX-b·oxalate).

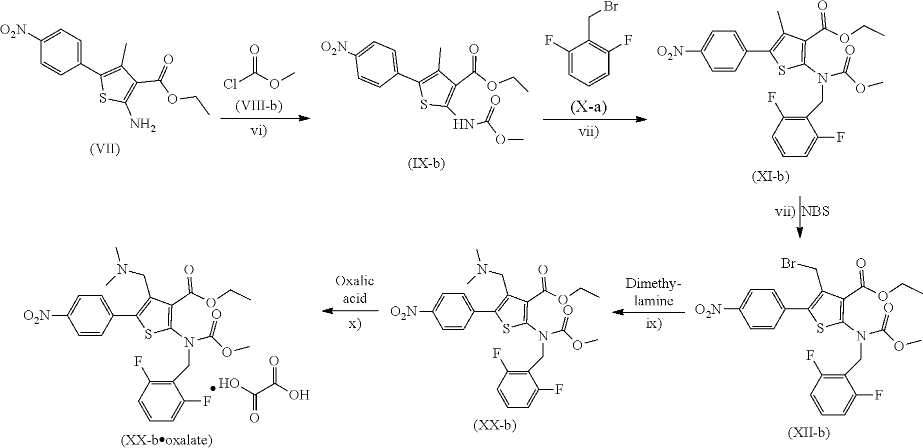

13. A compound of formula (XXVII-b·HCl).

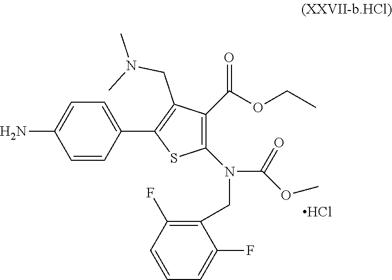 [SMILES: CCOC(=O)c1c(N(Cc2c(F)cccc2F)C(=O)OC)sc(-c2ccc(N)cc2)c1CN(C)C ; Cl]

14. A compound of formula (XX-b·oxalate).

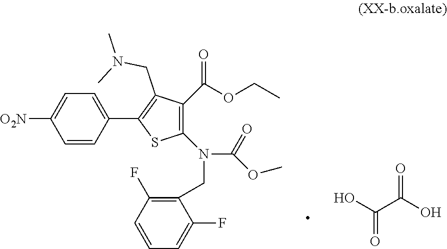 [SMILES: CCOC(=O)c1c(N(Cc2c(F)cccc2F)C(=O)OC)sc(-c2ccc([N+](=O)[O-])cc2)c1CN(C)C ; "·" ; O=C(O)C(=O)O]

15. The process according to claim 2, which previous to step a), further comprises the steps of:
j) coupling a compound of formula (XXVII-b·HCl) with methoxyamine or a salt thereof in the presence of a coupling agent and a suitable solvent to give a compound of formula (XXVIII-b), 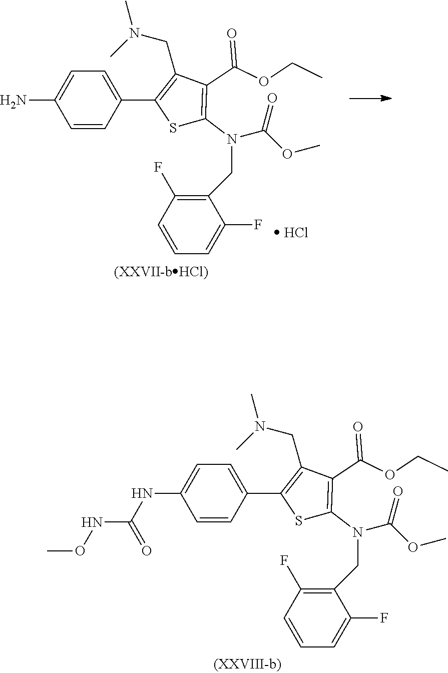
iv) hydrolyzing the compound of formula (XXVIII-b) obtained in step (i) to give a compound of formula (XXIX-b), and 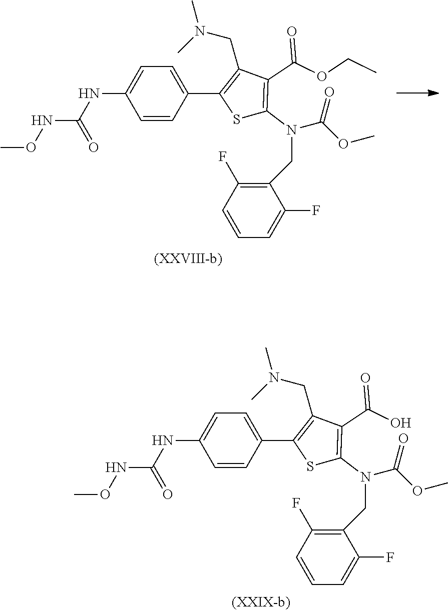
v) coupling the compound of formula (XXIX-b) obtained in step (ii) with 3-amino-6-methoxypyridazine of formula (XVIII) in the presence of a coupling agent and in a suitable solvent to give a compound of formula (XXVI-b).

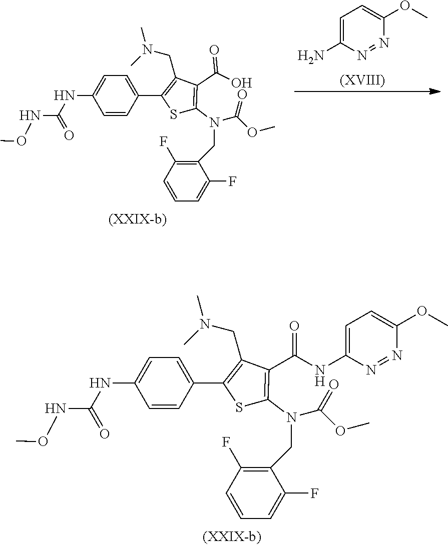

16. The process according to claim 15, wherein the methoxyamine is in the form of its hydrochloride salt.

17. The process according to claim 15, wherein the coupling agent in step i) is 1,1′-carbonyldiimidazole (CDI).

18. The process according to claim 15, wherein the hydrolysis agent in step ii) is sodium hydroxide.

19. The process according to claim 15, wherein the coupling agent in step iii) is propylphosphonic anhydride (T3P).

20. The process of claim 10, wherein the catalyst is palladium on charcoal (Pd/C).
